SEQUENCE LISTING (42 sequences)

SEQ ID NO: 1 - DNA, 1857 bp, Triticum aestivum
  source 1..1857: mol_type = genomic DNA
atggagagatcccgccgcctgctgctggtggcgggcctgctcgccgcgctgctcccggcggcggcggccgccttcgggcagcagccgggggcgccgtgcgagcccacgctgctggcgacgcaggtggcgctcttctgcgcgcccgacatgcccaccgcgcagtgctgcgagcccgtcgtcgccgccgtcgacctcggcggcggggtcccctgcctctgccgcgtcgccgcggagccgcagctcgtcatggcgggcctcaacgccacccacctcctcacgctctacagctcctgcggcggcctccgtcccggcggcgcccacctcgccgccgcctgcgaaggtacgcgcacgttcaccgccctccgtccctccctctctctgtctacgtgcagattttctgtgctctctttcctgcttgcctagtacgtagtgttccatggcctctcgggccgctagcgctccgatttgcgttggtttccttgctgttctgccggatctgttggcacggcgcgcggcgtcgggttctcgccgtctcccgtggcgagcgacctgcgcagcgcgcgcggcctggctagcttcataccgctgtaccttgagatacacggagcgatttagggtctactctgagtatttcgtcatcgtaggatgcatgtgccgctcgcgattgtttcatcgatttgagatctgtgcttgttcccgcgagttaagatggatctagcgccgtacgcagatgcagagtctgttgctcgagttaccttatctaccgtcgttcgactatggtatttgcctgcttccttttggctgggtttatcgtgcagtagtagtagacatgtggacgcgttcttcttattttgtgccgaccatcgtcgagatacttttcctgctacagcgtttcatcgcctgcaccatcccgttcgtgatagcacttttgtgtcaaaccgcaacgcagctttgctttctgcggtatcttctgccttgtttgtcgccttgcttggtcaaaactgagaactcttgctgtttgatcgaccgagggcagaggcagagcaagagcctgccgtgcttttggctctgcagtgcgtcgtctctgcctcctttgccaaacatttccatgttgatcctctgggggcactgctttttcgcatgcggtttccgtagccttcctctttcatgaaaaaaggtttgggtcaaatcaaatggatcgcctattggcagagcagcagcagatagctggctgtctcacagctttggcagaatcggtctgttgcctgccaccgtgtctcttatcttgcctgccaccgtgtctcttttcttgttgcgcacgtcgtcacctcctcctacttcttttccagttttgtttacttttgatgaaatacggacgaacggctggtaatcattaactttggttgctgttgttactgtggattttggacgcaggacccgctcccccggccgccgtcgtcagcagccccccgcccccgccaccgtcgaccgcacctcgccgcaagcagccagcgcgtgcgtacctctccctctcgcccgcatctcgctccgtattaactgattgtgtctgcatactgacgtgtgctttggctttggatctgtttcgcagacgacgcaccaccgccgccgccgccgtccagcgacaagccgtcgtccccgccgccgtcccaggaacacgacggcgccgctccccacgccaaggccgcccccgcccaggcggctacctccccgctcgcgcccgctgctgccatcgccccgccgccccaggcgccacactccgccgcgcccacggcgtcatccaaggcggccttcttcttcgtcgccacggccatgctcggcctctacatcatcctctga

SEQ ID NO: 2 - protein, 222 aa, Triticum aestivum
MERSRRLLLVAGLLAALLPAAAAAFGQQPGAPCEPTLLATQVALFCAPDMPTAQCCEPVVAAVDLGGGVPCLCRVAAEPQLVMAGLNATHLLTLYSSCGGLRPGGAHLAAACEGPAPPAAVVSSPPPPPPSTAPRRKQPAHDAPPPPPPSSDKPSSPPPSQEHDGAAPHAKAAPAQAATSPLAPAAAIAPPPQAPHSAAPTASSKAAFFFVATAMLGLYIIL

SEQ ID NO: 3 - DNA, 1856 bp, Triticum aestivum
  source 1..1856: mol_type = genomic DNA
atggagagatcccgcgggctgctgctggtggcggggctgctggcggcgctgctgccggcggcggcggcgcagccgggggcgccgtgcgagcccgcgctgctggcgacgcaggtggcgctcttctgcgcgcccgacatgccgacggcccagtgctgcgagcccgtcgtcgccgccgtcgacctcggcggcggggtgccctgcctctgccgcgtcgccgccgagccgcagctcgtcatggcgggcctcaacgccacccacctcctcacgctctacagctcctgcggcggcctccgccccggcggcgcccacctcgccgccgcctgcgaaggtacgttgtccgcctcctcccctccctccctccctccctctctctctacgtgctcgctttcctgcttacctagtagtacgtagtttcccatgccttcttgactcgctagaagtgctccggtttgggtctgttaatttcctcgctgtactaccggatctgtcgtcggcacggcgcgcggcgtcgggtcctcgccttctcccgtggcgaccgacctgcgcagcgcgcgcgcggcctagctagcttcataccgctgtacctcgacatacacggagcgatctatggtctactctgagtatttcctcatcgtagaacgcatgcgccgctcgcgattgtttcgtcgattctagatccgtgcttgttcccgcgagttagtatgcatctgcgtgcatatgccgtacgcacgcagatgcagagtctgttgctcgagttatctactgtcgttcgctcgaccatatttgcctgttaatttcctgttcatcgtgcatgcagtagtagtagccatgtccacgccttcttgttttgaggcgatcatcgtcgagatccatggctttgctttctgcactatcttctgccttgttttgttctccgcagtacgtacgtcttgcttggtcaaaactgaaaaacgctttgctgtttgtttgatcggcaagagctggccgtgcttttggcaccgcagtgcgtcgcctctgccgcttttgcgaaacatttccatgttgatcctctggcggaactactttttcgcgtgcggtttgcgtggccttcctctctcgtgaaaagaggtcgggtcaaaccaaatggatcgcctcttggcagagcagcggcagcagatagctggccgtctcgcagctttggcagaaccggtctgtggccatctgtcgccgcctgccaccgtttccctgatgtttgtttctctctcgcctgccactgtttcttttcttgttgcgcacgtacgtcgtcacctcctcctacttttttgccagttttgtttacttttgatgaaatatacggatgaatcggctggtgattaactttggctgctgctgttaattactgtggattttggatgcaggacccgctcccccggccgccgtcgtcagcagccccccgcccccgcctccaccgtccgccgcacctcgccgcaagcagccagcgcgtaagaacctctccctctccctctctctctccctctcgcctgcatctcgctatgtttatccatgtccatatgttgatcagccttgtttagttactaacatgtgcaccggatcgggttctcgcagacgacgcaccaccgccgccaccgccgtcgagcgagaagccgtcgtccccgccgccgtcccaggaccacgacggcgccgccccccgcgccaaggccgcgcccgcccaggcggccacctccacgctcgcgcccgccgccgccgccaccgccccgccgccccaggcgccgcactccgccgcgcccacggcgccgtccaaggcggccttcttcttcgtcgccacggccatgctcggcctctacatcatcctctga

SEQ ID NO: 4 - protein, 220 aa, Triticum aestivum
MERSRGLLLVAGLLAALLPAAAAQPGAPCEPALLATQVALFCAPDMPTAQCCEPVVAAVDLGGGVPCLCRVAAEPQLVMAGLNATHLLTLYSSCGGLRPGGAHLAAACEGPAPPAAVVSSPPPPPPPSAAPRRKQPAHDAPPPPPPSSEKPSSPPPSQDHDGAAPRAKAAPAQAATSTLAPAAAATAPPPQAPHSAAPTAPSKAAFFFVATAMLGLYIIL

SEQ ID NO: 5 - DNA, 1895 bp, Triticum aestivum
  source 1..1895: mol_type = genomic DNA
atggagagatcccgcggcctgctgctggcggcgggcctgctggcggcgctgctgccggcggcggcggccgcgttcgggcagcagccgggggcgccgtgcgagcccacgctgctggcgacgcaggtggcgctcttctgcgcgcccgacatgcccacggcccagtgctgcgagcccgtcgtcgccgccgtcgacctcggcggcggggtgccctgcctctgccgcgtcgccgcggagccgcagctcgtcatggcgggcctcaacgccacccacctcctcacgctctacggctcctgcggcggcctccgtcccggcggcgcccacctcgccgccgcctgcgaaggtacgtcgcgcacgttcaccgcctccctccctccctcgctctctctctctctctctctctctctctctacgtgccgattctctgtgttcgcttccctgcttacctagcacgtagttttccatggcttctcgactcgctggtcctccgatttgggtcggttaatttcctcgctgtactaccggatctgtcggcacggcgcgcggcgtcgggttctcgccgtctcccgtggcgagcgacctgcgcagcgcgcgcgcggcctagctagcttcataccgctgtaccttcagatacacggagcgatttagggtctactctgagtatttcgtcatcgtaggatgcatgtggcagtcgcgattgtttcatcgattttagatctgtgcttgttcccgcgagttaagatggatctagcgccgtacgcagacgcagatggtcttgctgtctctgttgctcgagttatcttatctactgtcgttcgagtatatttgcctgcttccttttgatctgtgtttatcgtgcagtagcagtagccatgtccacgccttcttgtttcgaggcgatcatcgtcgagatagcgctttgtttcaaaccgcaacgcagcctttgctttctgcggtatcttctgccttgtttttgttctgtgcagtacgtcttgcttggtcaaaagtaaaaactcttgctgttcgatcgaccgaggcctgatgcagagcaagagctggccgtgcttttcgctctgcagtgcatcgcctctgcctctttggccaaacatttccatgttgatcctctggtgtggtactacttttttgcatgcggtttgcgtagccttcctctttcgtgaaaaaaggtcgggtcgcctattggcagagcagcagcagcagcaacagatagctggctgtctcgcagctttgacagaaccggtctgtggccatctgtcgccgcctgccaccgtttccctgatgtttgtttctctcgtctcatctcgcctgccactgtttcttttcttgttgcgcacgtcgtcacctcctcctacttttttttccagttttgtttacttttgagatacggacgaacggctggtaattactaactttggttgctgttgttactgtggattttggacgcaggacccgctcccccggccgccatcgtcagcagccccccgcccccgccaccaccgtccgccgcacctcgccgcaagcagccagcgcgtacgaacctctccctccctctctctcgcctgcatctcgctctgtattagctgattgtgtttacttactgacgtgtgctttggctttggatctgtttcgcagacgacgcaccgccgccgccgccgccgtctagcgagaagccgtcgtccccgccgccgtcccaggagcacgacggcgccgccccccgcgccaaggccgcgcccgcccaggcgaccacctccccgctcgcgcccgctgccgccatcgccccgccgccccaggcgccacactccgcggcgcccacggcgtcgtccaaggcggccttcttcttcgtcgccacggccatgctcggcctctacatcatcctctga

SEQ ID NO: 6 - protein, 223 aa, Triticum aestivum
MERSRGLLLAAGLLAALLPAAAAAFGQQPGAPCEPTLLATQVALFCAPDMPTAQCCEPVVAAVDLGGGVPCLCRVAAEPQLVMAGLNATHLLTLYGSCGGLRPGGAHLAAACEGPAPPAAIVSSPPPPPPPSAAPRRKQPAHDAPPPPPPSSEKPSSPPPSQEHDGAAPRAKAAPAQATTSPLAPAAAIAPPPQAPHSAAPTASSKAAFFFVATAMLGLYIIL

SEQ ID NO: 7 - DNA, 1694 bp, Triticum aestivum
  source 1..1694: mol_type = genomic DNA
atggcccccaccgccctcctcatcgtcgtcctcgccgtcgctgccctccacgcccccgccgcctccgccgcgttgtcccaggagccaccagcgacgccgtgcgcggcagccatcgtgtccttctcgccgtgcctggcgcacgtcgcggtggtggcgccgcccgccctgccctcgcccgcgcccaccagcgcctgctgcgcggcgttcctgcgcgccgtttcctccggggacggcgaaggcggcggaggggagggatgcttctgccacctgctccgcaacccgctcctcctcggcttccccgtcgacgccgcgcgcctcggcactctcctccccacctgcgcctccgcgaaaacctccgccgccacggccgccgaggccgaggccctcttcgccgacaagtgccgaggtgagaaatctgtcccttcgtgctccctatttatgctcgtgcaatatgtatgcttcgatcaattctcgtgcgccatatgcgcgtgctgtcgcgttcctgttgttgatcgccgatcgaagcaaattttactctgcaaagtcctaactactgttgttctcatgatccttgtacaactactttcgtaatggttggttgtaatgcggattactgacgagctctgaatgctactagggatattagcacttgcattactgaactagtggaatgggggagaaacaccgcgggatttttcttttgttattaaggagaagatactgagtggcccagacttacttctgcgtttttgctgcgatttgtcatgattaccgttgatttaagcagtttgttgggttgcttgtttgatagtagtagtaatttcacaaaatattggcgatatttataaatagctaagggcttcgttacttggtggtttctctcaaagagttcacaaaagtcaattattaaattcaattaagggggcaagtattagtagtactggctaactccatttgccatttgccaattgaaacagagctcaagtcactgcctgagatgcattttacacctccatcgccacctcccgcaccaaaactttctccaggtaaatgttctgctgcttgtctaatgattccatagcttgttaaaaaaaatgattccataaatctgtgcccagtaatgctatttcggatttcggttgaatgaaccaattggcatttgggcagacatgtcatatgtcctctcctaccaaatgaaacttgaacttgttttatcttgtggtgctccatccatttcgtgttctatgccacaactgtacaggttcaaatgatagtagaacaactaattttgcatcggatactctctgcagcctctgttctgttcctactaattgcaggaccgagtctgtctttgtgatactatctgctttgcgtgcactgctcttttaggttgcagctgcaccttctattttgttcacctgagcattccattttggtcgactgaacaccgcaagtcctgaatatctttcttgaatcgtcggtcttacttggtgtgcactagttaaactgctgtgtatgcccttgcagctgccgttacagaaccagcgtccccgactccgaagatggaggagcattcgacctcgacgacgcctgtgtcggatgatcggtcgggatccgatgccttgtgtgcctgccgggtcttccttgtggccttggtcttgggagcagcagtcttgatcacgctgcagttctga

SEQ ID NO: 8 - protein, 213 aa, Triticum aestivum
MAPTALLIVVLAVAALHAPAASAALSQEPPATPCAAAIVSFSPCLAHVAVVAPPALPSPAPTSACCAAFLRAVSSGDGEGGGGEGCFCHLLRNPLLLGFPVDAARLGTLLPTCASAKTSAATAAEAEALFADKCRELKSLPEMHFTPPSPPPAPKLSPAAVTEPASPTPKMEEHSTSTTPVSDDRSGSDALCACRVFLVALVLGAAVLITLQF

SEQ ID NO: 9 - DNA, 1629 bp, Triticum aestivum
  source 1..1629: mol_type = genomic DNA
atgccccccaccgccctcctcctcgtcgtcctcgccgtcgccgccctccacgcccctgccgcctccgccgcgttgtcccaggagccaccagcgacgccgtgcgcggcggccatcgtgtccttctcgccgtgcctggcgccgtcgcggtggtggcgccgcccgccctgccctctcccgcgcccaccagcgcctgctgcgcggcgttcctgcgcgccgtttccgccggggacggcgaaggcggcggaggggagggatgcttctgccacctgctccgcgacccgctcctcctcggcttccccgtcgacgccgcgcgcctcggcgctctcctccccacctgcgcctccgcgaaaacctccgccgccacggccgtcgaggctgaggccctcttcgccgacaagtgccgaggtgagaaatctgtcccttcgtgctccctatttatgctcgtgcaatatgcatgcttcgatcaattctcgtgcgccatatgcgcgagctgtcgcgttcctgttgttgatcgccgatcgaaacaaatcttactctgcaaagccctaactactgttgttctcatgatccttgtacaactactttcgtaatggttggttgtaatgcggattactgacgagctctggatgctactagggatattagtacttgcatttctgaactagttgaatgggggagaaacaccgcgggatttttcttttgtggcccagacttacttctgcatttttgctgcgatttgtcatgattaccgttcagttaagcagtttgttgggttgcttgtttgatagtagtaatttcacaaaatattggcgatatttataaatagctaaaggcttcgttacttggtggtttctctcaaagagttcacaaaagtcaatcattaaatcaattaagggggcaagtattagtagtactggctaactccatttgccattgaaacagagctcaagtcactgcctgagatgcattttacacctccatcgccacctcccgcaccaaaactttctccaggtaaacgttctccctcttgtctaatgatttgataaatctgtgcccagtctattcgggtttcggttgaataaaccaattggcatttgggcagcatgttatatggtctctcctcatcccaaataaaacttgaacttgttttatcttgccgtgctccgtccattttgtgctctatgccgcaattgcacaggttcaatgatagtagaacacctagttttgcatcgcatactctctgcagcctctgttctgttcctgctaattgcagtatcgtgtctgtctttgtgatactatctcctttgcatgcactgcacttttaggttgcagttgcacgtttgattctagtttgttcacctgagcattccattttggtcgactgaacaccgcaagtcctgaatatctttcttgaatcgtcggtcgtacttcgtgtgcactagttaaactgctgtctatgcccttgcagctgccgttccagaaccagcgtccccgaagatggaggagcattcgacctcagcgacgcctgtgccggatgatcggtcgggatccgatgccttgtgtgcctgccgggtcttccttgtggccttggtcttgggagcagcagtcttgatcacgctgcagttctga

SEQ ID NO: 10 - protein, 205 aa, Triticum aestivum
MPPTALLLVVLAVAALHAPAASAALSQEPPATPCAAAIVSFSPCLAPSRWWRRPPCPLPRPPAPAARRSCAPFPPGTAKAAEGRDASATCSATRSSSASPSTPRASALSSPPAPPRKPPPPRPSRLRPSSPTSAESSSHCLRCILHLHRHLPHQNFLQLPFQNQRPRRWRSIRPQRRLCRMIGRDPMPCVPAGSSLWPWSWEQQS

SEQ ID NO: 11 - protein, 107 aa, Triticum aestivum
MAPTALLLVVLAVAALHAPAASAALSQEPPAMRDFSFSSSHCLRCILHLHRHLPHQNFLQLPFQNQRPRRWRSIRPQRRLCRMIGRDPMPCVPAGSSLWPWSWEQQS

SEQ ID NO: 12 - DNA, 1665 bp, Triticum aestivum
  source 1..1665: mol_type = genomic DNA
atggcccccaccgccctcctcctcgtcgtcctcgccgtcgccgccctccacgcccccgccgccaacgccgcgttgtcccaggagccaccagcgaccccgtgcgcggcggccatcgtgtccttctcgccgtgcctggcgcacgtcgcggtggtggcgccgcccgccctgccctcgcccgcgcccaccagcgcctgctgcgcggcgttcctgcgcgccgtttccgccggggacggcgaaggcggcggaggggagggatgcctctgccacctgctccgcgacccgctcctcctcggcttccccgtcgacgccgcgcgcctcggcgctctcctccccacctgcgcctccgcgaaaacctccgccgccacggccgtcgaggccgaggccctcttcgccgacaagtgccgaggtgagaaatctgtcccttcgtgctccctatttatgctcgtgcaatatgtatgctccgatcaattctcgtgcgccatatgcgcgtgctgtcgcgttcctgttgttgatcgccgaccgaaacaaattttactctgcaaagtcctaactactgttgttctcattatcctcgtacaactactttcgtaatggttggttgtaatgcggattactgacgagctctggatgctactagggatattagtacttgcatttctgaactagtggatgggggagaaagcgggatttttcttttgtggcccagacttacttctgcatttttgctgcgatttgtcatgattaccgttcagttaagcagtttgctgggttgcctgtttgatagtagtaatttcacaaaatattggcgatatttataaatagctaagggcttcgttacttggtggtttctctcaaagagttcacaaaagtcaatcattaaattcaattaagggggcaagtattagtagtactggctaactccatttgccatttgccaattgaaacagagctcaagtcactgcctgagatgcattttacacctccatcgccacctcccgcaccaaaactttctccaggtaaatgttctgctgcttgtctaatgattccatagctcgtcaaaaaaaaaatgattccataaatctgtgcccagtaatgctatttcggatttcggttgaatgaaccaattggcatttgggcagacatgtcatatttctttcctcccaaatgaaacttgaacttgttttatcttgtggtgctccatccatttcgtgctctatgccgcaactgtacaggttgaaatgatagtagaacatctaattttgcatcggatactctctgcagcctctgttctgtttctactaattgcaggatcgagtctgtctttgtgatactatctgctttgcgtgcactgctcttttaggttgcagctgcaccttctattttgttcacctgagcattccattttggtcgactgaacaccgcaagtcctgaatatctttcttgaatcgtcggtcgtacttcgtgtgcactagttaaactgctgtgtatgcccttgcagctgccgttccagaaccagcgtccccgactccgaagatggaggagcattcgacctcgacgacgtccatgtcggatgatcggtcgggatctgatgccttgtgtgcctgccgggtcttccttgtggccttggtcttgggagcagcagtcttgatcacgctgcagttctga

SEQ ID NO: 13 - protein, 213 aa, Triticum aestivum
MAPTALLLVVLAVAALHAPAANAALSQEPPATPCAAAIVSFSPCLAHVAVVAPPALPSPAPTSACCAAFLRAVSAGDGEGGGGEGCLCHLLRDPLLLGFPVDAARLGALLPTCASAKTSAATAVEAEALFADKCRELKSLPEMHFTPPSPPPAPKLSPAAVPEPASPTPKMEEHSTSTTSMSDDRSGSDALCACRVFLVALVLGAAVLITLQF

SEQ ID NO: 14 - DNA, 1666 bp, Triticum aestivum
  source 1..1666: mol_type = genomic DNA
atggcccccaccgccctcctcctcgtcgtcctcgccgtcgccgccctccacgcccccgccgccaacgccgcgttgtccccaggagccaccagcgaccccgtgcgcggcggccatcgtgtccttctcgccgtgcctggcgcacgtcgcggtggtggcgccgcccgccctgccctcgcccgcgcccaccagcgcctgctgcgcggcgttcctgcgcgccgtttccgccggggacggcgaaggcggcggaggggagggatgcctctgccacctgctccgcgacccgctcctcctcggcttccccgtcgacgccgcgcgcctcggcgctctcctccccacctgcgcctccgcgaaaacctccgccgccacggccgtcgaggccgaggccctcttcgccgacaagtgccgaggtgagaaatctgtcccttcgtgctccctatttatgctcgtgcaatatgtatgctccgatcaattctcgtgcgccatatgcgcgtgctgtcgcgttcctgttgttgatcgccgaccgaaacaaattttactctgcaaagtcctaactactgttgttctcattatcctcgtacaactactttcgtaatggttggttgtaatgcggattactgacgagctctggatgctactagggatattagtacttgcatttctgaactagtggatgggggagaaagcgggatttttcttttgtggcccagacttacttctgcatttttgctgcgatttgtcatgattaccgttcagttaagcagtttgctgggttgcctgtttgatagtagtaatttcacaaaatattggcgatatttataaatagctaagggcttcgttacttggtggtttctctcaaagagttcacaaaagtcaatcattaaattcaattaagggggcaagtattagtagtactggctaactccatttgccatttgccaattgaaacagagctcaagtcactgcctgagatgcattttacacctccatcgccacctcccgcaccaaaactttctccaggtaaatgttctgctgcttgtctaatgattccatagctcgtcaaaaaaaaaatgattccataaatctgtgcccagtaatgctatttcggatttcggttgaatgaaccaattggcatttgggcagacatgtcatatttctttcctcccaaatgaaacttgaacttgttttatcttgtggtgctccatccatttcgtgctctatgccgcaactgtacaggttgaaatgatagtagaacatctaattttgcatcggatactctctgcagcctctgttctgtttctactaattgcaggatcgagtctgtctttgtgatactatctgctttgcgtgcactgctcttttaggttgcagctgcaccttctattttgttcacctgagcattccattttggtcgactgaacaccgcaagtcctgaatatctttcttgaatcgtcggtcgtacttcgtgtgcactagttaaactgctgtgtatgcccttgcagctgccgttccagaaccagcgtccccgactccgaagatggaggagcattcgacctcgacgacgtccatgtcggatgatcggtcgggatctgatgccttgtgtgcctgccgggtcttccttgtggccttggtcttgggagcagcagtcttgatcacgctgcagttctga

SEQ ID NO: 15 - protein, 141 aa, Triticum aestivum
MAPTALLLVVLAVAALHAPAANAALSPGATSDPVRGGHRVLLAVPGARRGGGAARPALARAHQRLLRGVPARRFRRGRRRRRRGGMPLPPAPRPAPPRLPRRRRAPRRSPPHLRLRENLRRHGRRGRGPLRRQVPRAQVTA

SEQ ID NO: 16 - DNA, 1525 bp, Triticum aestivum
  source 1..1525: mol_type = genomic DNA
atggggcggccgccgtgctgcgacaaggccaacgtgaagaaggggccgtggacggcggaggaggacgccaagctgctggcctacacctccaaccatggcaccggcaactggacctctgttccccagagggcaggtcggaaccctctcccccggccggccggatcggcgtctggcatcgaaatggtgttggttttgcatggtttctggctgatgtgtgttgcgtcatggatgggtgcgtgctgcagggctgaagcggtgcgggaagagctgcaggctgaggtacaccaactacctgaggcccaacctcaagcacgagaacttcacgcaggaggaggaggagctcatcgtcaccctccatgccatgctgggaagcaggtgcatctctgtacatgcatgtcatccatcggttactttctcgattttgtcatcaagttttcggtgaatgcgaaactttgctatggacagactactgtttctgtcatcattcctgctataaactttgcatccatatactctagcgtacgttcagatcttatagatttatggaagttctaaaaacctcacgttggtgtatcattcagacaattgctcatacaccggaaatcacgtcctacttctattcaagaatttactttagccgttcattcataaagtagaaacaaatgtagtttaatccagtaaccagtctcgtgtctacggttcttctgaatatattctgtttcctggtagaatgtagtgaaaagctgaatattgggtgaagaacagatctgaatagctgtcttaaattgatagtgtttgacagaaaatactccacactgagcgtaatataacatgaaagtacgtgtacgtgttgggtgcatgcaggtggtctctgatcgcgaaccagctgccggggcggacggacaacgacgtcaagaattactggaacaccaagctgagcaagaagctgcggcagcggggcatcgaccccatcacccatcgccccatcgccgacctcatgcagagcatcggcaccctccccatccgcccgccgcccagcgccgcgggtgcctcctcgtcctcctacatccccgtgaacccagcggcggcgccggggctccagccgctgcacgacgacgtcaaataccacgcagtcctgaaccaccaccagcagcaggtcatcacgctcctcgaccccgacgcgccaggggcggcggcgtccccggaccaccagctcaagtggagcgacttccttgccgacgacgccgccgccttcgaggcggcgccgcaggtggttcttggtcagtaccaggaggccgcggtggctggtggcggagcagcgtatggagacactgatagtattgcagccgatggtgtcggcgggggcggggaggatagcgcagcgtcagcgttcatcgacgcgatgctggacagcgacaaaaagatgggcgtggaccagctcatcgccgacctgctcgccgacccggcatactactacggcggaggctcttcctcttcgacgtcggagctggggcggggcggttga

SEQ ID NO: 17 - protein, 307 aa, Triticum aestivum
MGRPPCCDKANVKKGPWTAEEDAKLLAYTSNHGTGNWTSVPQRAGLKRCGKSCRLRYTNYLRPNLKHENFTQEEEELIVTLHAMLGSRWSLIANQLPGRTDNDVKNYWNTKLSKKLRQRGIDPITHRPIADLMQSIGTLPIRPPPSAAGASSSSYIPVNPAAAPGLQPLHDDVKYHAVLNHHQQQVITLLDPDAPGAAASPDHQLKWSDFLADDAAAFEAAPQVVLGQYQEAAVAGGGAAYGDTDSIAADGVGGGGEDSAASAFIDAMLDSDKKMGVDQLIADLLADPAYYYGGGSSSSTSELGRGG

SEQ ID NO: 18 - DNA, 1530 bp, Triticum aestivum
  source 1..1530: mol_type = genomic DNA
atggggcggccgccgtgctgcgacaaggccaacgtgaagaaggggccgtggacggcggaggaggacgccaagctgctcgcctacacctccaaccatggcaccggcaactggacctctgttccccagagggcaggtcggaaccctccccccggccggccggatcgacgtccgtcatggaaatggtgttggtcttgcatggtttctggctgatgtgtgttgcatcatggatgcgtgcgtgctgcagggctgaagcggtgcgggaagagctgcaggctgaggtacaccaactacctgaggcccaacctcaagcacgagaacttcacgcaggaggaggaggagctcatcgtcaccctccatgccatgctgggaagcaggtgcgtctctctgtgtagtacatgcatgtttctgccatcagttactttctcgattttgtcatcaagttttcggtgaatgcgaaactttgctatggaccgactgctgtttctgtcatcattcctgctataaaccttgcatccatatactctagcgttcagatcttatagatttatggaagttctgaatatctcacgttggtgtatcagacaattgctcatataccggaaatcacgtcctacttctattcaagaatttacttcagtcgttcgttcaaaaatagaaagaaatgtagtttagtccagtgaccagtctcgtgtctacgtacggttcttctgaatatatatattctgtttcctggtagaatgtagtgaaaagctgaaaattcggtgaagaacatatctgaatagctgtcttaaattgatagtgtttgacagaaaatactccacactgagcgtaacgtgaaagtgcgtgtacgtgttgggtgcatgcaggtggtctctgatcgcgaaccagctgccggggcggacggacaacgacgtcaagaactactggaacaccaagctgagcaagaagctgaggcagcggggcatcgaccccatcacccaccgccccatcgccgatctcatgcagagcatcggcaccctcgccatccgcccgccaccgagcgccgcgggtgcctcctcctcctcctacctccccgtgaacccaccggcggcgccggggctccagccgctgcacgacgacgtcaaataccacacagtcctgaaccagcagcagcagcaggtcatcacgctcctcgaccccgacgcgccaggggcggcggcgtccccggagcaccagctcaagtggagcgacttcctcgcggacgacgccgcggccctcgaggcggcgccgcaggtcgttcttggtcagtaccaggaggccgcggtcgctggtggcggagcacacgcgtatggcgacactgacagtactgcagccgatggtgtcggcgggggcggggacgatagcgcagcgtcagcgttcatcgacgcgatgctggacagcgacaagaagatgggcgtggaccagctcatcgccgacctgctcgccgacccggcatactactacggcggaggctcttcctcttcgacgtcggagctggggtggggctgttga

SEQ ID NO: 19 - protein, 308 aa, Triticum aestivum
MGRPPCCDKANVKKGPWTAEEDAKLLAYTSNHGTGNWTSVPQRAGLKRCGKSCRLRYTNYLRPNLKHENFTQEEEELIVTLHAMLGSRWSLIANQLPGRTDNDVKNYWNTKLSKKLRQRGIDPITHRPIADLMQSIGTLAIRPPPSAAGASSSSYLPVNPAAAPGLQPLHDDVKYHAVLNQQQQQVITLLDADAPGAAASPDHPLKWSDFLADDAAAFEAAPQVVLGQYQDAAVAGGGAHAYGDTDSTAADGVGVGGEDSAASAFIDAMLDSDKKMGVDQLIADLLADPAYYYGGGSSSSTSDLGWGC

SEQ ID NO: 20 - DNA, 1530 bp, Triticum aestivum; identical to SEQ ID NO: 18
  source 1..1530: mol_type = genomic DNA

SEQ ID NO: 21 - protein, 308 aa, Triticum aestivum
MGRPPCCDKANVKKGPWTAEEDAKLLAYTSNHGTGNWTSVPQRAGLKRCGKSCRLRYTNYLRPNLKHENFTQEEEELIVTLHAMLGSRWSLIANQLPGRTDNDVKNYWNTKLSKKLRQRGIDPITHRPIADLMQSIGTLAIRPPPSAAGASSSSYLPVNPPAAPGLQPLHDDVKYHTVLNQQQQQVITLLDPDAPGAAASPEHQLKWSDFLADDAAALEAAPQVVLGQYQEAAVAGGGAHAYGDTDSTAADGVGGGGDDSAASAFIDAMLDSDKKMGVDQLIADLLADPAYYYGGGSSSSTSELGWGC

SEQ ID NO: 22 - DNA, 414 bp, Triticum aestivum
  source 1..414: mol_type = genomic DNA
atgttgaggatgcagcagcaggtggagggcgtggtgggcggcggcatcgtggccgaggcggaggaggcggcggtgtacgagcgggtggctcgcatggccagcggcaacgccgtggtcgtcttcagcgccagcggctgctgcatgtgccacgtcgtcaagcgcctcctgcttggcctgggagtcggccccaccgtctacgagttggaccagatgggcggcgccgggcgggagatccaggcggccctggcgcagctgctgccccccggacccggcgccggccaccaccagcagccgccggtgcccgtggtgttcgtcggcgggaggctcctgggaggcgtggagaaggtgatggcgtgccacatcaacggcacgctcgtcccgctcctcaaggacgccggcgcgctctggctctga

SEQ ID NO: 23 - protein, 137 aa, Triticum aestivum
MLRMQQQVEGVVGGGIVAEAEEAAVYERVARMASGNAVVVFSASGCCMCHVVKRLLLGLGVGPTVYELDQMGGAGREIQAALAQLLPPGPGAGHHQQPPVPVVFVGGRLLGGVEKVMACHINGTLVPLLKDAGALWL

SEQ ID NO: 24 - DNA, 414 bp, Triticum aestivum
  source 1..414: mol_type = genomic DNA
atgttgaggatgcagcagcaggtggagggcgtggtgggcggcggcatcgtggcggaggcggaggaggcggcggtgtacgagagggtggctcgcatggccagcggcaacgcggtggtcgtcttcagcgccagcggctgctgcatgtgccacgtcgtcaagcgcctcctgcttggcctgggagtcggccccaccgtgtacgagttggaccagatgggcggcgccgggcgggagatccaggcggccctggcgcagctgctgccccccggacccggcgccggccaccaccagcagccgccggtgcccgtggtgttcgttggcgggaggctcctgggcggcgtggagaaggtgatggcgtgccacatcaacggcacgctcgtcccgctcctcaaggacgccggcgcgctctggctctga

SEQ ID NO: 25 - protein, 137 aa, Triticum aestivum; identical to SEQ ID NO: 23

SEQ ID NO: 26 - DNA, 414 bp, Triticum aestivum
  source 1..414: mol_type = genomic DNA
atgttgaggatgcagcagcaggtggagggcgtggtgggcggcggcatcatggcggaggcggaggaggcggcggtgtacgagcgggtggctcgcatggccagcggcaacgcggtggtcgtcttcagcgccagcggctgctgcatgtgccacgtcgtcaagcgcctcctgcttggcctgggggtcggccccaccgtctacgagttggaccagatgggcggcgccgggcgagagatccaggcggcgctggcgcagctgctgccccccggacccggcgccggccaccaccagcagccgccagtgcccgtggtgttcgtcggcgggaggctcctgggcggcgtggagaaggtgatggcgtgccacatcaacggcacgctcgtcccgctcctcaaggacgccggcgcgctctggctctga

SEQ ID NO: 27 - protein, 137 aa, Triticum aestivum
MLRMQQQVEGVVGGGIMAEAEEAAVYERVARMASGNAVVVFSASGCCMCHVVKRLLLGLGVGPTVYELDQMGGAGREIQAALAQLLPPGPGAGHHQQPPVPVVFVGGRLLGGVEKVMACHINGTLVPLLKDAGALWL

SEQ ID NO: 28 - DNA, 1926 bp, Triticum aestivum
  source 1..1926: mol_type = genomic DNA
atgagcagccccatggaggaagctcaccatggcatgccgtcgacgacgacggcgttcttcccgctggcagggctccacaagttcatggccatcttcctcgtgttcctctcgtggatcttggtccactggtggagcctgaggaagcagaaggggccgaggtcatggccggtcatcggcgcgacgctggagcagctgaggaactactaccggatgcacgactggctcgtggagtacctgtccaagcaccggacggtcaccgtcgacatgcccttcacctcctacacctacatcgccgacccggtgaacgtcgagcatgtgctcaagaccaacttcaacaattaccccaaggtgaaactgaaagaacccctcagccttgtgaatttttttgccaaggttcagaagtttacactgacacaaatgtctgaaattgtacgtgtagggggaggtgtacaggtcctacatggacgtgctgctcggcgacggcatcttcaacgccgacggcgagctctggaggaagcagaggaagacggcgagcttcgagttcgcttccaagaacctgagagactttagcacgatcgtgttcagggagtactccctgaagctgcgcagcatcctgagccaggcttgcaaggccggcaaagtcgtggacatgcaggtaaccgaactcagtcccttggtcatctgaacattgatttcttggacaaaatttcaagattctgacgcgagcgagcgaattcaggagctgtacatgaggatgacgctggactcgatctgcaaggtggggttcggggtcgagatcggcacgctgtcgccggagctgccggagaacagcttcgcgcaggcgttcgacgccgccaacatcatcgtgacgctgcggttcatcgacccgctgtggcgcgtgaagaagttcctgcacgtcggctcggaggcgctgctggagcagagcatcaagctcgtcgacgagttcacctacagcgtcatccgccggcgcaaggccgagatcgtgcaggcccgggccagcggcaagcaggagaaggtgcgtacgtgatcgtcgtcgtcaagctccggatcgctggtttgtgtaggtgccattgatcactgacacactagctgggtgcgcagatcaagcacgacatactgtcgcggttcatcgagctgggcgaggccggcggggacgacggcggcagcctgttcggggacgacaagggcctccgcgacgtggtgctcaacttcgtgatcgccgggcgggacaccacggccacgacgctctcctggttcacctacatggccatgacgcacccggccgtggccgagaagctccgccgcgagctggccgccttcgaggcggaccgcgcccgcgaggatggcgtcgcgctggtcccctgcagcgactcagacggcgacggctccgacgaggccttcgccgcccgcgtggcgcagttcgcggggctgctgagctacgacgggctcgggaagctggtgtacctccacgcgtgcgtgacggagacgctgcgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccggagcggtggatcggcgacgacggcgcgttccgcaacgcgtcgccgttcaagttcacggcgttccaggcggggccgcggatctgcctcggcaaggactcggcgtacctgcagatgaagatggcgctggccatcctgtgcaggttcttcaggttcgagctcgtggagggccaccccgtcaagtaccgcatgatgaccatcctctccatggcgcacggcctcaaggtccgcgtctccagggcgccgctcgcctga

SEQ ID NO: 29 - protein, 554 aa, Triticum aestivum
MSSPMEEAHHGMPSTTTAFFPLAGLHKFMAIFLVFLSWILVHWWSLRKQKGPRSWPVIGATLEQLRNYYRMHDWLVEYLSKHRTVTVDMPFTSYTYIADPVNVEHVLKTNFNNYPKGEVYRSYMDVLLGDGIFNADGELWRKQRKTASFEFASKNLRDFSTIVFREYSLKLRSILSQACKAGKVVDMQELYMRMTLDSICKVGFGVEIGTLSPELPENSFAQAFDAANIIVTLRFIDPLWRVKKFLHVGSEALLEQSIKLVDEFTYSVIRRRKAEIVQARASGKQEKIKHDILSRFIELGEAGGDDGGSLFGDDKGLRDVVLNFVIAGRDTTATTLSWFTYMAMTHPAVAEKLRRELAAFEADRAREDGVALVPCSDSDGDGSDEAFAARVAQFAGLLSYDGLGKLVYLHACVTETLRLYPAVPQDPKGIAEDDVLPDGTKVRAGGMVTYVPYSMGRMEYNWGPDAASFRPERWIGDDGAFRNASPFKFTAFQAGPRICLGKDSAYLQMKMALAILCRFFRFELVEGHPVKYRMMTILSMAHGLKVRVSRAPLA

SEQ ID NO: 30 - DNA, 1920 bp, Triticum aestivum
  source 1..1920: mol_type = genomic DNA
atgagcagccccatggaggaagctcaccttggcatgccgtcgacgacggccttcttcccgctggcagggctccacaagttcatggccgtcttcctcgtgttcctctcgtggatcctggtccactggtggagcctgaggaagcagaaggggccacggtcatggccggtcatcggcgcgacgctggagcagctgaggaactactaccggatgcacgactggctcgtggagtacctgtccaagcaccggacggtcaccgtcgacatgcccttcacctcctacacctacatcgccgacccggtgaacgtcgagcacgtgctcaagaccaacttcaacaattaccccaaggtgaaacaatcctcgagatgtcagtcaaggttcagtataatcggtactgacagtgttacaaatgtctgaaatctggaattgtgtgtgtagggggaggtgtacaggtcctacatggacgtgctgctcggcgacggcatattcaacgccgacggcgagctctggaggaagcagaggaagacggcgagcttcgagttcgcttccaagaacttgagagacttcagcacgatcgtgttcagggagtactccctgaagctgtccagcatcctgagccaggcttgcaaggcaggcaaagttgtggacatgcaggtaactgaactctttcccttggtcatatgaacgttgatttcttggacaaaatctcaagattctgacgcgagcgagccaattcaggagctgtacatgaggatgacgctggactcgatctgcaaggtggggttcggggtggagatcggcacgctgtcgccggagctgccggagaacagcttcgcgcaggccttcgacgccgccaacatcatcgtgacgctgcggttcatcgacccgctgtggcgcgtgaagaaattcctgcacgtcggctcggaggcgctgctggagcagagcatcaagctcgtcgacgagttcacctacagcgtcatccgccggcgcaaggccgagatcgtgcaagcccgggccagcggcaagcaggagaaggtgcgtacgtggtcatcgtcattcgtcaagctcccgatcgctggtttgtgcagatgccactgatcactgacacattaactgggcgcgcagatcaagcacgacatactgtcgcggttcatcgagctgggcgaggccggcggcgacgacggcggcagcctgttcggggacgacaagggcctccgcgacgtggtgctcaacttcgtcatcgccgggcgggacacgacggccacgacgctctcctggttcacctacatggccatgacgcacccggccgtggccgagaagctccgccgcgagctggccgccttcgagtccgagcgcgcccgcgaggatggcgtcgctctggtcccctgcagcgacggcgagggctccgacgaggccttcgccgcccgcgtggcgcagttcgcgggactcctgagctacgacgggctcgggaagctggtgtacctccacgcgtgcgtgacggagacgctccgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccagagcggtggatcggcgacgacggcgccttccgcaacgcgtcgccgttcaagttcacggcgttccaggcggggccgcggatctgcctgggcaaggactcggcgtacctgcagatgaagatggcgctggccatcctgtgcaggttcttcaggttcgagctcgtggagggccaccccgtcaagtaccgcatgatgaccatcctctccatggcgcacggcctcaaggtccgcgtctccagggtgccgctcgcctga

SEQ ID NO: 31 - protein, 551 aa, Triticum aestivum
MSSPMEEAHLGMPSTTAFFPLAGLHKFMAVFLVFLSWILVHWWSLRKQKGPRSWPVIGATLEQLRNYYRMHDWLVEYLSKHRTVTVDMPFTSYTYIADPVNVEHVLKTNFNNYPKGEVYRSYMDVLLGDGIFNADGELWRKQRKTASFEFASKNLRDFSTIVFREYSLKLSSILSQACKAGKVVDMQELYMRMTLDSICKVGFGVEIGTLSPELPENSFAQAFDAANIIVTLRFIDPLWRVKKFLHVGSEALLEQSIKLVDEFTYSVIRRRKAEIVQARASGKQEKIKHDILSRFIELGEAGGDDGGSLFGDDKGLRDVVLNFVIAGRDTTATTLSWFTYMAMTHPAVAEKLRRELAAFESERAREDGVALVPCSDGEGSDEAFAARVAQFAGLLSYDGLGKLVYLHACVTETLRLYPAVPQDPKGIAEDDVLPDGTKVRAGGMVTYVPYSMGRMEYNWGPDAASFRPERWIGDDGAFRNASPFKFTAFQAGPRICLGKDSAYLQMKMALAILCRFFRFELVEGHPVKYRMMTILSMAHGLKVRVSRVPLA

SEQ ID NO: 32 - DNA, 1920 bp, Triticum aestivum
  source 1..1920: mol_type = genomic DNA
atgagcagccccatggaggaagctcacggcggcatgccgtcgacgacggccttcttcccgctggcagggctccacaagttcatggccatcttcctcgtgttcctctcgtggatcttggtccactggtggagcctgaggaagcagaaggggccgaggtcatggccggtcatcggcgcgacgctggagcagctgaggaactactaccggatgcacgactggctcgtggagtacctgtccaagcaccggacggtgaccgtcgacatgcccttcacctcctacacctacatcgccgacccggtgaacgtcgagcatgtgctcaagaccaacttcaacaattaccccaaggtgaaacaatcctcgagatgtcagtaaaggttcagtataatcggtactgacagtgttacaaatgtctgaaatctgaaattgtatgtgtagggggaggtgtacaggtcctacatggacgtgctgctcggcgacggcatattcaacgccgacggcgagctctggaggaagcagaggaagacggcgagcttcgagttcgcttccaagaacttgagagacttcagcacgatcgtgttcagggagtactccctgaagctgtccagcatactgagccaggcttgcaaggccggcaaagttgtggacatgcaggtaactgaactcattcccttggtcatctgaacgttgatttcttggacaaaatttcaagattctgacgcgagcgagcgaattcaggagctgtatatgaggatgacgctggactcgatctgcaaagtggggttcggagtcgagatcggcacgctgtcgccggagctgccggagaacagcttcgcgcaggcgttcgacgccgccaacatcatcgtgacgctgcggttcatcgacccgctgtggcgcgtgaagaagttcctgcacgtcggctcggaggcgctgctggagcagagcatcaagctcgtcgacgagttcacctacagcgtcatccgccggcgcaaggccgagatcgtgcaggcccgggccagcggcaagcaggagaaggtgcgtgcgtggtcatcgtcattcgtcaagctcccggtcgctggtttgtgtagatgccatggatcactgacacactaactgggcgcgcagatcaagcacgacatactgtcgcggttcatcgagctgggcgaggccggcggcgacgacggcggcagtctgttcggggacgacaagggcctccgcgacgtggtgctcaacttcgtgatcgccgggcgggacaccacggccacgacgctgtcctggttcacctacatggccatgacgcacccggacgtggccgagaagctccgccgcgagctggccgccttcgaggcggagcgcgcccgcgaggatggcgtcgctctggtcccctgcggcgacggcgagggctccgacgaggccttcgctgcccgcgtggcgcagttcgcggggttcctgagctacgacggcctcgggaagctggtgtacctccacgcgtgcgtgacggagacgctgcgcctgtacccggcggtgccgcaggaccccaagggcatcgcggaggacgacgtgctcccggacggcaccaaggtgcgcgccggcgggatggtgacgtacgtgccctactccatggggcggatggagtacaactggggccccgacgccgccagcttccggccggagcggtggatcggcgacgacggcgccttccgcaacgcgtcgccgttcaagttcacggcgttccaggcggggccgcggatttgcctcggcaaggactcggcgtacctgcagatgaagatggcgctggcaatcctgtgcaggttcttcaggttcgagctcgtggagggccaccccgtcaagtaccgcatgatgaccatcctctccatggcgcacggcctcaaggtccgcgtctccagggcgccgctcgcctga

SEQ ID NO: 33 - protein, 551 aa, Triticum aestivum
MSSPMEEAHGGMPSTTAFFPLAGLHKFMAIFLVFLSWILVHWWSLRKQKGPRSWPVIGATLEQLRNYYRMHDWLVEYLSKHRTVTVDMPFTSYTYIADPVNVEHVLKTNFNNYPKGEVYRSYMDVLLGDGIFNADGELWRKQRKTASFEFASKNLRDFSTIVFREYSLKLSSILSQACKAGKVVDMQELYMRMTLDSICKVGFGVEIGTLSPELPENSFAQAFDAANIIVTLRFIDPLWRVKKFLHVGSEALLEQSIKLVDEFTYSVIRRRKAEIVQARASGKQEKIKHDILSRFIELGEAGGDDGGSLFGDDKGLRDVVLNFVIAGRDTTATTLSWFTYMAMTHPDVAEKLRRELAAFEAERAREDGVALVPCGDGEGSDEAFAARVAQFAGFLSYDGLGKLVYLHACVTETLRLYPAVPQDPKGIAEDDVLPDGTKVRAGGMVTYVPYSMGRMEYNWGPDAASFRPERWIGDDGAFRNASPFKFTAFQAGPRICLGKDSAYLQMKMALAILCRFFRFELVEGHPVKYRMMTILSMAHGLKVRVSRAPLA

SEQ ID NO: 34 - DNA, 1483 bp, Triticum aestivum
  source 1..1483: mol_type = genomic DNA
atggaagagaagaagccgcggcggcagggagccgcaggacgcgatggcatcgtgcagtacccgcacctcttcatcgcggccctggcgctggccctggtcctcatggaccccttccacctcggcccgctggccgggatcgactaccggccggtgaagcacgagctggcgccgtacagggaggtcatgcagcgctggccgagggacaacggcagccgcctcaggctcggcaggctcgagttcgtcaacgaggtgttcgggccagagtccatcgagttcgaccgccagggccgcgggccctacgccgggctcgccgacggccgcgtcgtgcggtggatgggggacaaggccgggtgggagacgttcgccgtcatgaatcctgactggtattggcttactgcagaaaaaccatagcttacctgtgtgtgtgcaaactaaaatagttttttcggaaaaaaaaaggtcggagaaagtttgtgctaacggagtggagtcgacgacgaagaagcagcacgggaaggagaagtggtgcggccggcctctcgggctgaggttccacagggagaccggcgagctcttcatcgccgacgcgtactatgggctcatggccgttggcgaaagcggcggcgtggcgacctccctggcgagggaggccggcggggacccggtccacttcgccaacgacctcgacatccacatgaacggctcgatattcttcaccgacacgagcacgagatacagcagaaagtgagcggagtactgctgccgatctcctttttctgttcttgagatttgtgtttgacaaatgactgatcatgcagggaccatttgaacattttgctggaaggagaaggcacggggaggctgctgagatatgaccgagaaaccggtgccgttcatgtcgtgctcaacgggctggtcttcccaaacggcgtgcagatctcacaggaccagcaatttctcctcttctccgagacaacaaactgcaggtgagataaactcaggttttcagtatgatccggctcgagagatccaggaactgatgacgcctttattaatcggctcatgcatgcacactaggatcatgaggtactggctggaaggtccaagagcgggccaggtggaggtgttcgcgaacctgccggggttccccgacaacgtgcgcttgaacagcaaggggcagttctgggtggcgatcgactgctgccggacgccgacgcaggaggtgttcgcgcggtggccgtggctgcggaccgcctacttcaagatcccggtgtcgatgaagacgctggggaagatggtgagcatgaagatgtacacgcttctcgcgctcctcgacggcgaggggaacgtggtcgaggtactcgaggaccggggcggcgaggtgatgaagctggtgagcgaggtgagggaggtggaccggaggctgtggatcgggaccgttgcgcacaaccacatcgccacgatcccttatccgttggactag

SEQ ID NO: 35 - protein, 413 aa, Triticum aestivum
MEEKKPRRQGAAGRDGIVQYPHLFIAALALALVLMDPFHLGPLAGIDYRPVKHELAPYREVMQRWPRDNGSRLRLGRLEFVNEVFGPESIEFDRQGRGPYAGLADGRVVRWMGDKAGWETFAVMNPDWSEKVCANGVESTTKKQHGKEKWCGRPLGLRFHRETGELFIADAYYGLMAVGESGGVATSLAREAGGDPVHFANDLDIHMNGSIFFTDTSTRYSRKDHLNILLEGEGTGRLLRYDRETGAVHVVLNGLVFPNGVQISQDQQFLLFSETTNCRIMRYWLEGPRAGQVEVFANLPGFPDNVRLNSKGQFWVAIDCCRTPTQEVFARWPWLRTAYFKIPVSMKTLGKMVSMKMYTLLALLDGEGNVVEVLEDRGGEVMKLVSEVREVDRRLWIGTVAHNHIATIPYPLD

SEQ ID NO: 36 - DNA, 1481 bp, Triticum aestivum
  source 1..1481: mol_type = genomic DNA
atggaagagaagaagccgcggcggcagggagccgcagtacgcgatggcatcgtgcagtacccgcacctcttcatcgcggccctggcgctggccctggtcgtcatggaccccttccacctcggcccgctggctgggatcgactaccggccggtgaagcacgagctggcgccatacagggaggtcatgcagcgctggccgagggacaacggcagccgcctcaggctcggcaggctcgagttcgtcaacgaggtgttcgggccggagtccatcgagttcgacagccagggccgcgggccctacgccgggctcgccgacggccgcgtcgtgcggtggatgggggacaagaccgggtgggagacgttcgccgtcatgaatcctgactggtaattggcttactgcagataaatccatagcttacctgtgtgtttgcaaactaaaatgatttcttgggaaaaaaaaaggtcggagaaagtttgtgctaacggagtggagtcaacgacgaagaagcagcacgggaaggagaagtggtgcggccggcctctcgggctgaggttccacagggagaccggcgagctcttcatcgccgacgcgtactatgggctcatggccgtcggcgaaagcggcggcgtggcgacctccctggcaagggaggtcggcggggacccggtccacttcgccaacgacctcgacatccacatgaacggctcgatattcttcaccgacacgagcacgagatacagcagaaagtgagcggagtactgtcgctgatctccatttttgttcttgagatgttgtgtttgagtgtctgacaccatgactgatcatgcagggatcatttgaacattttgctagaaggagaaggcacggggaggctgctgagatatgaccgagaaaccggtgccgttcatgtcgtgctcaacgggctggtcttcccaaacggcgtgcagatttcacaggaccagcaatttctcctcttctccgagacaaccaactgcaggtgagataaactcaggttttcagtatgatccggctcgagagatccaggaactgatgacggatcatgcatgcacgctaggatcatgaggtactggctggaaggtccaagagcgggccaggtggaggtgttcgcgaacctgccggggttccccgacaacgtgcgcctgaacagcaaggggcagttctgggtggcgatcgactgctgccggacgccgacgcaggaggtgttcgcgaggtggccgtggctgcggaccgcctacttcaagatcccggtgtcgatgaagacgctggggaagatggtgagcatgaagatgtacacgcttctcgcgctcctcgacggcgaggggaacgtggtggaggtgctcgaggaccggggcggcgaggtgatgaagctggtgagcgaggtgagggaggtggaccggaggctgtggatcgggaccgttgcgcacaaccacatcgccacgatcccttacccgctggactag

SEQ ID NO: 37 - protein, 413 aa, Triticum aestivum
MEEKKPRRQGAAVRDGIVQYPHLFIAALALALVVMDPFHLGPLAGIDYRPVKHELAPYREVMQRWPRDNGSRLRLGRLEFVNEVFGPESIEFDSQGRGPYAGLADGRVVRWMGDKTGWETFAVMNPDWSEKVCANGVESTTKKQHGKEKWCGRPLGLRFHRETGELFIADAYYGLMAVGESGGVATSLAREVGGDPVHFANDLDIHMNGSIFFTDTSTRYSRKDHLNILLEGEGTGRLLRYDRETGAVHVVLNGLVFPNGVQISQDQQFLLFSETTNCRIMRYWLEGPRAGQVEVFANLPGFPDNVRLNSKGQFWVAIDCCRTPTQEVFARWPWLRTAYFKIPVSMKTLGKMVSMKMYTLLALLDGEGNVVEVLEDRGGEVMKLVSEVREVDRRLWIGTVAHNHIATIPYPLD

SEQ ID NO: 38 - DNA, 1471 bp, Triticum aestivum
  source 1..1471: mol_type = genomic DNA
atggaagagaagaaaccgcggcggcagggagccgcagtacgcgatggcatcgtgcagtacccgcacctcttcatcgcggccctggcgctggccctggtcctcatggacccgttccacctcggcccgctggccgggatcgactaccgaccggtgaagcacgagctggcgccgtacagggaggtcatgcagcgctggccgagggacaacggcagccgcctcaggctcggcaggctcgagttcgtcaacgaggtgttcgggccggagtccatcgagttcgaccgccagggccgcgggccttacgccgggctcgccgacggccgcgtcgtgcggtggatgggggacaaggccgggtgggagacgttcgccgtcatgaatcctgactggtactggcttactgcagaaaaacccatagcttacctgtgtgtgtgcagactaaaatagtttctttcataaaaaaaaggtcggagaaagtttgtgctaacggagtggagtcgacgacgaagaagcagcacgggaaggagaagtggtgcggccggcctctcggcctgaggttccacagggagaccggcgagctcttcatcgccgacgcgtactatgggctcatggccgtcggcgaaaggggcggcgtggcgacctccctggcgagggaggccggcggggacccggtccacttcgccaacgaccttgacatccacatgaacggctcgatattcttcaccgacacgagcacgagatacagcagaaagtgagcggagtactgctgccgatctcctttttctgttcttgagatttgtgtttgacaaatgactgatcatgcagggaccatttgaacattttgctggaaggagaaggcacggggaggctgctgagatatgaccgagaaaccggtgccgttcatgtcgtgctcaacgggctggtcttcccaaacggcgtgcagatatcacaggaccagcaatttctcctcttctccgagacaacaaactgcaggtgagataaactcaggttttcagtatgatccggctcgagagatccaggaactgatgacggctcatgcatgcacactaggatcatgaggtactggctggaaggtccaagagcgggccaggtggaggtgttcgcgaacctgccggggttccccgacaatgtgcgcctgaacagcaaggggcagttctgggtggccatcgactgctgccgtacgccgacgcaggaggtgttcgcgcggtggccgtggctgcggaccgcctacttcaagatcccggtgtcgatgaagacgctggggaagatggtgagcatgaagatgtacacgcttctcgcgctcctcgacggcgaggggaacgtcgtggaggtgctcgaggaccggggcggcgaggtgatgaagctggtgagcgaggtgagggaggtggaccggaggctgtggatcgggaccgttgcgcacaaccacatcgccacgatcccttacccgctggactag

SEQ ID NO: 39 - protein, 413 aa, Triticum aestivum
MEEKKPRRQGAAVRDGIVQYPHLFIAALALALVLMDPFHLGPLAGIDYRPVKHELAPYREVMQRWPRDNGSRLRLGRLEFVNEVFGPESIEFDRQGRGPYAGLADGRVVRWMGDKAGWETFAVMNPDWSEKVCANGVESTTKKQHGKEKWCGRPLGLRFHRETGELFIADAYYGLMAVGERGGVATSLAREAGGDPVHFANDLDIHMNGSIFFTDTSTRYSRKDHLNILLEGEGTGRLLRYDRETGAVHVVLNGLVFPNGVQISQDQQFLLFSETTNCRIMRYWLEGPRAGQVEVFANLPGFPDNVRLNSKGQFWVAIDCCRTPTQEVFARWPWLRTAYFKIPVSMKTLGKMVSMKMYTLLALLDGEGNVVEVLEDRGGEVMKLVSEVREVDRRLWIGTVAHNHIATIPYPLD

SEQ ID NO: 40 - DNA, 20 bp, synthetic construct
  misc_feature 1..20: note = synthesized
  source 1..20: mol_type = other DNA
ggaggtacaccaactacctg

SEQ ID NO: 41 - protein, 568 aa, Thinopyrum ponticum
MREIATQRCGNRSMALSAPPSQEQPSGKQFGYQLAAAVRSINWTYGIFWSISASPRPGHSSVLAWKDGFYNGEIKTRKITGSTTTELTADERVMHRSKQLRELYESLLPGNSNNRARRPTASLSPEDLGDGEWYYTISMTYTFHPNQGLPGKSFASNQHVWLYNAQYANTRVFPRALLAKTIVCIPFMGGVLELGTSDQVLEDPSMVKRISTSFWELHLPSSLESKDPSSSTSANDTREATDIILFEDFDHNDTVEGVISEQREVQCPSNVNLERLTKQMDEFHSLLGGLDVHPLEDRWIMDEPFEFTFSPEVAPAMDMPSTDDVIVTLSRSEGSRPSCFTAWKGSSESKYVAGQVVGESQKLLNKVVAGGAWASNYGGRTMVRAQGINSNTHVMTERRRREKLNEMFLVLKSLVPSIHKVDKASILTETIGYLRELKQRVDQLESSRSPSHPKETTGPSRSHVVGARKKIVSAGSKRKAPGLESPSNVVNVTMLDKVVLLEVQCPWKELLMTQVFDAIKSLCLDVVSVQASTSGGRLDLKIRANQQLAVGSAMVAPGAITETLQKAI

SEQ ID NO: 42 - protein, 573 aa, Thinopyrum ponticum
MREIATQRCGNRSMALSAPPSQEQPSGKQFGYQLAAAVRSINWTYGIFWSISASPRPGHSSVLAWKDGFYNGEIKTRKITGSTTTELTADERVMHRSKQLRELYESLLPGNSNNRARRPTASLSPEDLGDGEWYYTISMTYTFHPNQGLPGKSFASNQHVWLYNAQYANTRVFPRALLAKTASIQTIVCIPFMGGVLELGTSDQVLEDPSMVKRINTSFWELHLPSSLESKDPSSSTSANDTREATDIILFEDFDHNDTVEGVISEQREVQCTSNVNLERLTKQMDEFHSLLGGLDVHPLKDRWIMDEPFEFTFSPEVAPAMDMPSTDDVIVTLSRSEGSRPSCFTAWKGSSESKYVAGQVVGESQKLLNKVVAGGAWASNYGGRTMVRAQGINSNTHVMTERRRREKLNEMFLVLKSLVPSIHKVDKASILTETIGYLRELKQRVDQLESSRSPSHPKETTGPSRSHVVGARKKIVSAGSKRKAPGLESPSNVVNVTMLDKVVLLEVQCPWKELLMTQVFDAIKSLCLDVVSVQASTSGGRLDLKIRANQQLAVGSAMVAPGAITETLQKAI

(19) United States
(12) Patent Application Publication
(10) Pub. No.: US 2025/0027106 A1
(43) Pub. Date: Jan. 23, 2025

(54) FERTILITY RESTORATION IN PLANTS

(71) Applicant: PIONEER HI-BRED INTERNATIONAL, INC., JOHNSTON, IA (US)

(72) Inventors: MARC C. ALBERTSEN, GRIMES, IA (US); TIM FOX, DES MOINES, IA (US); MANJIT SINGH, JOHNSTON, IA (US); MARK E. WILLIAMS, JOHNSTON, IA (US)

(73) Assignee: PIONEER HI-BRED INTERNATIONAL, INC., JOHNSTON, IA (US)

(21) Appl. No.: 18/910,454

(22) Filed: Oct. 9, 2024

Related U.S. Application Data
(63) Continuation of application No. 17/275,111, filed on Mar. 10, 2021, now Pat. No. 12,146,148.
(60) Provisional application No. 62/832,716, filed on Apr. 11, 2019.
(60) Provisional application No. 62/815,261, filed on Mar. 7, 2019.
(60) Provisional application No. 62/731,252, filed on Sep. 14, 2018.

(51) Int. Cl. C12N 15/82

(52) U.S. Cl. CPC C12N 15/8289; C12N 15/8213

(57) ABSTRACT

Provided herein are methods and compositions for restoring fertility and maintaining sterility in plants. In particular, one method disclosed includes introducing into a male-sterile plant, wherein the plant comprises one or more homozygous mutations in a male-fertility gene, a plant restoration donor chromosomal component comprising a plant-derived polynucleotide that confers a plant phenotypic marker linked to a male-fertility restoration locus that functionally complements the male-sterility phenotype from the one or more homozygous mutations in the male-sterile plant. In some examples, the plant-derived polynucleotide that confers a plant phenotypic marker and the male-fertility restoration locus are linked to each other and located on the same chromosomal arm on the plant restoration donor chromosomal component. In some examples, the plant-derived polynucleotide that confers a plant phenotypic marker, the male-fertility restoration locus, or both are modified.

### CROSS REFERENCE TO RELATED APPLICATIONS

This application claims the benefit of U.S. Provisional Application No. 62/832,716, filed Apr. 11, 2019; U.S. Provisional Application No. 62/815,261, filed Mar. 7, 2019; and U.S. Provisional Application No. 62/731,252, filed Sep. 14, 2018; the entire contents of each is herein incorporated by reference.

### FIELD

The present invention relates to the field of plant molecular biology, more particularly to impacting male fertility.

### REFERENCE TO SEQUENCE LISTING SUBMITTED ELECTRONICALLY

The official copy of the sequence listing is submitted electronically via EFS-Web as an ASCII formatted sequence listing with a file named 7795WOPCT_seqlisting_ST25.txt, produced on Sep. 8, 2019, and having a size of 97 kilobytes and is filed concurrently with the specification. The sequence listing contained in this ASCII formatted document is part of the specification and is herein incorporated by reference in its entirety.

### BACKGROUND

Development of hybrid plant breeding has made possible considerable advances in quality and quantity of crops produced. Increased yield and combination of desirable characteristics, such as resistance to disease and insects, heat and drought tolerance, along with variations in plant composition are all possible because of hybridization procedures. These procedures frequently rely heavily on providing for a male parent contributing pollen to a female parent to produce the resulting hybrid.

Field crops are bred through techniques that take advantage of the plant's method of pollination. A plant is self-pollinating if pollen from one flower is transferred to the same or another flower of the same plant or a genetically identical plant. A plant is cross-pollinated if the pollen comes from a flower on a different plant.

In certain species, such as Brassica campestris, the plant is normally self-sterile and can only be cross-pollinated. In self-pollinating species, such as soybeans, cotton and wheat, the male and female plants are anatomically juxtaposed. During natural pollination, the male reproductive organs of a given flower pollinate the female reproductive organs of the same flower. Maize has male flowers, located on the tassel, and female flowers, located on the ear, on the same plant and can be bred by both self-pollination and cross-pollination techniques,

During hybrid seed production, it is desirable to prevent self-pollination of the female inbred to avoid production and harvesting of female inbred seeds, since they exhibit less vigor than the hybrid seeds. To increase commercial quantities of the resulting hybrid seed, hybrid seed is often obtained using male-sterile female parents and male parents.

### SUMMARY

Provided herein are compositions and methods of restoring male fertility in a male-sterile plant. Male-fertility may be restored to the male-sterile plant by providing a plant restoration donor chromosomal component from a different species or plant than the male-sterile plant. The plant restoration donor chromosomal component contains a male-fertility restoration locus that functionally complements the male-sterility phenotype of a plant rendered male-sterile due to one or more homozygous recessive mutations in a male-fertility polynucleotide. The male-fertility restoration locus is linked to a plant-derived polynucleotide that confers a plant phenotypic marker. Also provided herein are plants, plant cells, and seeds that have one or more homozygous mutations in a male-fertility polynucleotide and contain the plant restoration donor chromosomal component. In some examples, the plant restoration donor chromosomal component plant substitutes for a chromosome native to the male-sterile plant so that the plant produces euploid seed.

In some embodiments disclosed herein, plant restoration donor chromosomal components are modified with respect to one or more plant-derived polynucleotides that confer a plant phenotypic marker and/or one or more male-fertility restoration loci. For example, a modified plant-derived polynucleotides and/or male-fertility restoration locus may be modified in its polynucleotide sequence, copy number, expression level, or location within the plant restoration donor chromosomal component as compared to a native, non-modified plant-derived polynucleotide that confers a plant phenotypic marker or male-fertility restoration locus. Also disclosed herein are plants, plant cells, and seeds having these plant restoration donor chromosomal components.

Accordingly, described herein are methods for making and using such plants. Maintainer plants having any of the plant restoration donor chromosomal components described herein may be used to produce seed by allowing them to self-fertilize. The seeds, plants, or parts thereof containing the plant restoration donor chromosomal component may be identified using the plant phenotypic marker, for example, in seed sorting. Absence of the plant phenotypic marker's expression in the seeds indicates that the seeds do not contain the plant restoration donor chromosomal component and, when planted, will give rise to male-sterile female plants. Conversely, the presence of the plant phenotypic marker in seeds indicates that the seeds contain the plant restoration donor chromosomal component and, when planted, will give rise to male-fertile plants. Such seeds and plants may be used in the maintenance of male-sterility, male-sterile female inbreds for hybrid and seed increase production.

### BRIEF DESCRIPTION OF THE FIGURES

FIG. 1 shows examples of phenotypes in F3 generation in wheat. Part A) shows blue-colored wheat seed that will give rise to male-fertile wheat plants and part B) shows white-colored (non-blue colored) seed that will give rise to male-sterile female wheat plants.

FIG. 2. shows an example of seed from a wheat F3 plant segregating for seed color with a portion of white-colored wheat seeds that will give rise to male-sterile female wheat plants, a portion of light blue-colored wheat seeds (heterozygous for blue aleurone) that will give rise to male-fertile wheat plants; and a portion of dark blue-colored wheat seeds (homozygous for blue aleurone) that will give rise to male-fertile wheat plants.

FIG. 3 illustrates one embodiment of a pericentric inversion of Ms1 to increase linkage with BA (blue aleurone) on a chromosome.

FIG. 4 shows one embodiment of a translocation and replacement of a wheat chromosomal arm with a plant restoration donor chromosomal component of 4E-Ms45. Part A shows a plant restoration chromosomal component of a 4EL telosomic addition chromosome that provides Ms45 and BA. Part B shows a plant restoration donor chromosomal component of 4EL that provides Ms45 and BA and translocates and replaces a 5AL wheat chromosome.

### DETAILED DESCRIPTION

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Provided herein are plants, plant parts, plant cells or seeds having a plant restoration donor chromosomal component that can be used to restore male-fertility to male-sterile plants and facilitate the identification of plant parts, plant cells or seeds having the plant restoration donor chromosomal component. As used herein, a “plant restoration donor chromosomal component” is a chromosome or fragment thereof that includes a plant-derived polynucleotide that confers a plant phenotypic marker linked to a male-fertility restoration locus. As used herein, the term “plant-derived” indicates that the polynucleotide that confers the plant phenotypic marker is from a plant. In some examples, the plant-derived polynucleotide that confers the plant phenotypic marker is endogenous with respect to the plant restoration donor chromosomal component and male-fertility restoration locus. As used herein, the term “endogenous” or “native” or “natively” means normally present in the specified plant, present in its normal state or location in the chromosome (non-modified), plant cell, or plant.

In some examples, the plant-derived polynucleotide is exogenous with respect to the plant restoration donor chromosomal component, plant, or plant cell into which it is being introduced. The term “exogenous” means not normally present in the chromosomal component, plant, plant cell; not present in its normal state or location in the chromosomal component, plant, or plant cell, is introduced into the chromosomal component, plant cell or plant, or originates from a different chromosome, type of plant, plant species, or, if from the same chromosome, type of plant, plant species, is in a different location, modified from its native form in composition and/or genomic locus by deliberate human intervention. In some examples, the plant restoration donor chromosomal component includes one or more plant-derived polynucleotides that confer the plant phenotypic marker including, but not limited to, one or more native, edited, repositioned, replaced, or inserted plant-derived polynucleotides that confer the plant phenotypic marker or combinations thereof. In embodiments where there are two or more plant-derived polynucleotides, the polynucleotides may be the same or different from one another, for example, with respect to their sequences, such as their origin, e.g. plant type or species, polynucleotide or amino acid sequence or location in the plant restoration donor chromosomal component. In some examples, the one or more plant-derived polynucleotides that confer the plant phenotypic marker is exogenous with respect to the plant, plant cell, or plant restoration donor chromosomal component into which it is being introduced or combinations thereof.

Expression of the plant phenotypic marker in seed allows for the seed to be identified, selected, and/or sorted from seeds that do not contain the plant restoration donor chromosomal component, i.e. not having the plant-derived polynucleotide that confers the plant phenotypic marker. In some aspects, the plant phenotypic marker is a non-destructive marker.

The plant phenotypic marker may relate to the color, physiology, or morphology of the plant or seed. Examples of seed phenotypes that are suitable markers include but are not limed to seed color, seed color intensity or pattern, seed shape, seed surface texture, seed size including seed size width and/or length, seed density, or other seed characteristics. Examples of seed phenotypic color markers include but are not limited to blue aleurone, P gene that regulates flavonoid synthesis in maize, anthocyanin, Kala 4, and other endosperm coloring traits. In some examples, the plant-derived polynucleotide is ThMYC4E and confers a blue aleurone phenotype. See, Li, Na et al. “ThMYC4E, Candidate Blue Aleurone 1 Gene Controlling the Associated Trait in Triticum aestivum.” Ed. Harsh Raman. PLoS ONE 12.7 (2017): e0181116. PMC. Web. 13 Sep. 2018, herein incorporated by reference in its entirety. See, published PCT patent application, WO2019090496, herein incorporated by reference in its entirety and in the instant sequence listing. In some embodiments where there are two or more plant-derived polynucleotides, the two plant-derived polynucleotides are polynucleotides encoding blue alerone color markers that confer a blue aleurone phenotype to the seed and may be used for seed identification, selection, and sorting. The color markers be the same or different from one another, for example, with respect to their sequences, such as their origin, e.g. plant type or species, polynucleotide or amino acid sequence or location in the plant restoration donor chromosomal component.

As desired, the plant-derived polynucleotides that confer the plant phenotypic marker may be modified to increase its expression in the plant, for example, to increase the expression of the plant phenotypic marker in a plant, plant part thereof, or seed. In some aspects, the regulatory region of the plant-derived polynucleotide may be modified to increase expression of the plant phenotypic marker, for example, by editing the existing regulatory region to replace, delete, and/or insert nucleotides for improved expression. See, for example, PCT Patent publication WO2018183878, published Oct. 4, 2018, incorporated herein by reference in its entirety Alternatively, or in addition to, nucleotides in the plant phenotypic marker on the plant restoration donor chromosomal component may be modified to change the polynucleotide so it uses codons preferred by the host plant.

In some embodiments, the blue aleurone gene in the plant restoration chromosomal component has a nucleic acid sequence encoding an amino acid sequence with at least 80%, at least 85%, at least 90%, at least 95%), at least 96%, at least 97%, at least 98%, or at least 99% sequence identity to the amino acid sequence of SEQ ID NO: 41 or 42, variants, or fragments thereof. The nucleic acid sequence encoding the polypeptide that confers the blue aleurone phenotype may be synthesized, isolated, or obtained from any number of sources, including but not limited to, Agropyron, Thinopyrum, or Triticum, such as Agropyron elongatum, Agropyron trichophorum, Triticum thaoudar, Triticum aestivum, Triticum monococcum, and Thinopyrum ponticum. The source of blue aleurone and the plant restoration donor chromosomal component may be from Sebesta Blue, Blue Sando, Blue Baart, Blue Onas, Blue 1, PBB, or Blue Norco, or other addition line that comprises a polynucleotide encoding blue aleurone. The presence of blue aleurone in the seed may be indicated by the appearance of a blue color in the aleurone layer of the seed, confirmed using PCR, or any other suitable assays. The examples of plant phenotypic markers included herein are not meant to be limiting. Any desired plant phenotypic marker may be used in the methods and compositions described herein.

Seeds may be sorted into various populations using any suitable seed phenotypic marker. For example, the absence of the plant phenotypic marker in the seed, e.g. seed lacking the plant restoration donor chromosomal component, indicates the seed, when planted, will give rise to a male-sterile female plant. Plants from this seed may be used as male-sterile female inbreds for hybrid and seed increase production. The presence of the plant phenotypic marker in the seed, e.g. seed having the plant restoration donor chromosomal component, indicates that the seed will give rise to a male-fertile plant that may be used as a maintainer for the male-sterile female plant. As discussed elsewhere herein, the seeds may be sorted using any suitable approach or instrument so long as it has sufficient sensitivity to detect the difference between phenotypic marker expressing and non-expressing seeds.

In addition to the plant-derived polynucleotide that confers the plant phenotypic marker, the plant restoration donor chromosomal component includes a male-fertility restoration locus that is able to functionally complement the male-sterility phenotype of a plant. As used herein, a “male-fertility restoration locus” refers to one or more male-fertility polynucleotides that, when expressed in a male-sterile female plant, restores the plant to male-fertility by complementing the female plant's male sterile condition caused by one or more homozygous mutations in a male-fertility polynucleotide. Such mutations may be a substitution, a deletion, and/or an insertion of one or more nucleotides that results in conferring male-sterility to a plant. As used herein, the term “male-fertility polynucleotide” means one of the polynucleotides critical to a specific step in microsporogenesis, the term applied to the entire process of pollen formation. In some examples, the one or more male-fertility polynucleotides include but are not limited to Ms1, Ms5, Ms9, Ms22, Ms26, or Ms45. In some embodiments, the male-fertility restoration locus utilizes without limitation two or more male-fertility polynucleotides of Ms1, Ms5, Ms9, Ms22, Ms26, or Ms45 to complement a female plant's male sterile condition caused by one or more homozygous mutations in the Ms1, Ms5, Ms9, Ms22, Ms26, or Ms45 male-fertility polynucleotide, respectively.

As desired, the male-fertility restoration locus may be modified to increase expression of the male-fertility polynucleotide in the plant, for example, to complement male-sterility or restore male-fertility to a male-sterile plant. In some aspects, the regulatory region may be modified to increase expression of the male-fertility restoration locus or male-fertility polynucleotide, for example, by editing the existing regulatory region to replace, delete, and/or insert nucleotides for improved expression. See, for example, PCT Patent publication WO2018183878, published Oct. 4, 2018, incorporated herein by reference in its entirety. Alternatively, or in addition to, nucleotides in the male-fertility restoration locus or male-fertility polynucleotide on the plant restoration donor chromosomal component may be modified to change the polynucleotide so it uses codons preferred by the host plant.

In some examples, the male-fertility restoration locus is associated with increased male-fertile phenotype in a plant. The plant's male-fertility condition can be assessed by any suitable technique, for example, by observation of the plant's male tissue development, such as phenotyping of anthers and seed set on individual plants. See, for example, Example 1 herein.

In some examples, the male-fertility restoration locus includes, but is not limited to, one or more native, edited, replaced, repositioned, or inserted male-fertility polynucleotides or combinations thereof. In embodiments where there are two or more male-fertility polynucleotides in the male-fertility restoration loci, the male-fertility restoration loci may be the same or different from one another, for example, with respect to their sequences, such as their origin, e.g. plant type or species, polynucleotide or amino acid sequence or location in the plant restoration donor chromosomal component. In some aspects, the one or more male-fertility restoration locus is endogenous with respect to the plant, plant cell, or plant restoration donor chromosomal component into which it is being introduced or combinations thereof. In some aspects, the one or more male-fertility restoration locus is exogenous with respect to the plant, plant cell, or plant restoration donor chromosomal component into which it is being introduced or combinations thereof. The male-fertility restoration may include one or more male-fertility polynucleotides known to one skilled in the art, described herein, including homologs and orthologs of any the foregoing.

In one example, the male-fertility restoration locus functionally complements the male-sterility phenotype from one or more homozygous mutations in an endogenous male-fertility polynucleotide that confers male-sterility to the plant. This includes but is not limited to one or more homozygous recessive alleles for Ms1, Ms5, Ms9, Ms22, Ms26, or Ms45. Since Ms1 in wheat behaves as a single gene recessive, in some embodiments, only the ms1 male-fertility polynucleotide or allele located on chromosome 4BS may need to be mutated to confer male-sterility to a wheat plant.

There are a number of known male-fertility polynucleotides and male-fertility mutants from wheat and other species, including but not limited to Ms1, Ms5, Ms9, Ms22, Ms26, or Ms45.

PCT Patent publication WO2016048891, published Mar. 31, 2016, describes a male fertility gene referred to as “MS1” that is located on wheat chromosome 4BS and encodes a glycosylphosphatidylinositol (GPI)-anchored nsLTP (LTPG) polypeptide (also referred to as TaLTPGI) important to male fertility. Examples of DNA and polypeptide sequences of wheat Ms1 sequences are disclosed in WO2016048891, published Mar. 31, 2016, and WO2019118342, published Jun. 20, 2019, each of which are incorporated herein in its entirety, and in the instant sequence listing.

Wheat Ms5 is a glycosylphosphatidylinositol-anchored lipid transfer protein required for normal pollen exine development and the gene is located on wheat chromosome 3A. Examples of DNA and polypeptide sequences of wheat Ms5 are disclosed in WO2019118342, published Jun. 20, 2019, each of which are incorporated herein in its entirety, and in the instant sequence listing.

U.S. Patent publication US20150191743 A1, published Jul. 9, 2015, describes a male fertility gene referred to as “MS9” that is located on maize chromosome 1 and encodes a myb transcription factor critical to male fertility. The Ms9 phenotype was first identified in maize in 1932. Beadle, (1932) Genetics 17:413-431. It was found to be linked to the P1 gene on Chromosome 1. Breakdown of male reproductive tissue development occurs very early in premeiosis; tapetal cells may be affected as well. Greyson, et al., (1980) Can. J. Genet. Cytol. 22:153-166. Examples of genomic DNA and polypeptide sequences of maize Ms9 are disclosed in US patent publication 20150191743, published on Jul. 9, 2015, incorporated herein in its entirety. Wheat Ms9 is located on the long arm of wheat chromosome 4. Examples of genomic DNA and polypeptide sequences of wheat Ms9 are disclosed in US patent publication US20190177722A1, published on Jun. 13, 2019, incorporated herein in its entirety, and in the instant sequence listing.

U.S. Patent publication US20090038026A1, published Feb. 5, 2009, describes a male fertile gene referred to as “Msca1” or “MS22” that is located on maize chromosome 7 and encodes a protein critical to male fertility. Mutations referred to as ms22 or msca1 were first noted as phenotypically male sterile with anthers which did not extrude from the tassel and lacked sporogenous tissue. West and Albertsen (1985) Maize Newsletter 59:87; Neuffer et al. (1977) Mutants of maize. Cold Spring Harbor Laboratory Press, Cold Spring Harbor, NY. The mutant locus was originally referred to as ms22 but was later changed to msca1, or male sterile converted anther. See Chaubal et al. “The transformation of anthers in the msca1 mutant of maize” Planta (2003)216:778-788. Wheat Ms22 is located on the long arm of wheat chromosome 2. Examples of genomic DNA and polypeptide sequences of wheat Ms22 are disclosed in US patent publication US20190177722A1, published on Jun. 13, 2019, incorporated herein in its entirety, and in the instant sequence listing.

U.S. Pat. No. 7,517,975, issued Apr. 14, 2009, describes a male fertility gene referred to as “MS26” (also known as SB200 or SBMu200) that is located on maize chromosome 1. Ms26 sequences in maize or rice, e.g. as disclosed in U.S. Pat. Nos. 7,919,676 or 8,293,970. In wheat, the Ms26 gene is located on wheat chromosome 4AS. Examples of genomic DNA and polypeptide sequences of wheat Ms26 are disclosed in US patent publication US20190177722A1, published on Jun. 13, 2019, incorporated herein in its entirety, and in the instant sequence listing.

U.S. Pat. No. 5,478,369 issued Dec. 26, 1995 describes a male fertile gene referred to as “MS45” cloned on maize chromosome 9. In wheat, the Ms45 gene is located on the long arm of wheat chromosome 4. Examples of genomic DNA and polypeptide sequences of wheat Ms45 are disclosed in US patent publication US20190177722A1, published on Jun. 13, 2019, incorporated herein in its entirety, and in the instant sequence listing.

Male-sterile female plants for use in the methods and compositions described herein can be generated using any number of methods recognized in the art, including but not limited to mutagenesis, suppression, and genome editing. Mutations that cause male sterility in crop plants such as maize, wheat and rice have been produced by a variety of methods such as X-rays or UV-irradiations, chemical treatments, or transposable element insertions (Chaubal et al. 2000) Am J Bot 87:1193-1201). Suppression technology such as antisense, co-suppression, RNAi, hairpin formations may be used to disrupt or prevent expression of the fertility gene alleles to create a male-sterile female plant, e.g. to create homozygous recessive alleles that confer male-sterility to the plant. In some instances, male-sterility results from using genome editing technology to introduce a genetic modification (mutation) near or into an endogenous male-fertility gene, e.g. a male-fertility polynucleotide, to cause sterility. See, WO2015026883, published on Feb. 26, 2015, and Singh, M., Kumar, M., Albertsen, M. C. et al. Plant Mol Biol (2018) 97: 371-383. Accordingly, the methods may employ CRISPR technology using a guide RNA/Cas endonuclease system, where the Cas endonuclease is guided by the guide RNA to recognize and optionally introduce a double strand break at a specific target site into the plant genome of a cell. In some examples, the wheat genomes (A, B, and D) contain homologous genes that have similar gene structure and function, requiring triple mutants to result in a male-sterile phenotype, e.g. one, two, or three homozygous mutations in a male-fertility polynucleotide. In some embodiments, the male-sterile phenotype is caused by the introduction of genetic modification (mutation) of a target site located in or near one or more endogenous male fertility gene locus of Ms1, Ms5, Ms9, Ms22, Ms26 or Ms45 in a plant cell's genome. See, for example, US20190177722A1, published on Jun. 13, 2019, and WO2019118342, published Jun. 20, 2019, each of which are incorporated herein in its entirety.

Accordingly, known or created ms1/ms1/; ms5/ms5; ms9/ms9/; ms22/ms22/; ms26/ms26/; or ms45/ms45 male-sterile edited or mutant plants may be used in the methods and compositions described herein, for example, for use as male-sterile female plants in hybrid and hybrid seed production. Since Ms1 in wheat behaves as a single gene recessive, in some embodiments only the ms1 male-fertility polynucleotide or allele located on chromosome 4BS may need to be mutated to confer male-sterility to a wheat plant.

In some embodiments, provided herein are compositions and methods to complement and restore male fertility to male-sterile female wheat plants containing one or more homozygous mutations in a Ms1, Ms5, Ms9, Ms22, Ms26 or Ms45 male-fertility polynucleotide which confer male-sterility to the wheat plant. In some examples, the male-sterile plant contains one or more homozygous mutations in a recessive sporophytic male fertility polynucleotide. In some examples, these male-sterile wheat plants may be restored to male fertility when a plant restoration donor chromosomal component from a non-wheat species is used to functionally complement the one or more homozygous Ms1, Ms5, Ms9, Ms22, Ms26 or Ms45 mutations that confer male-sterility to the wheat plant.

In some embodiments, the wheat Ms1 male-fertility polynucleotide sequences include (a) a polynucleotide comprising the sequence set forth in SEQ ID NO:1, 3, or 5; (b) a polynucleotide having at least 85%, 90% or 95% sequence identity to SEQ ID NO: 1, 3, or 5; (c) a polynucleotide that encodes a polypeptide having at least 85%, 90% or 95% sequence identity to SEQ ID NO:2, 4, or 6; and (d) a polynucleotide that encodes a polypeptide of SEQ ID NO: 2, 4, or 6.

In some embodiments, the wheat Ms5 male-fertility polynucleotide sequences include (a) a polynucleotide comprising the sequence set forth in SEQ ID NO:7, 9, 12, or 14; (b) a polynucleotide having at least 85%, 90% or 95% sequence identity to SEQ ID NO: 7, 9, 12, or 14; (c) a polynucleotide that encodes a polypeptide having at least 85%, 90% or 95% sequence identity to SEQ ID NO: 8, 10, 11, 13, or 15; and (d) a polynucleotide that encodes a polypeptide of SEQ ID NO: 8, 10, 11, 13, or 15.

In some embodiments, the wheat Ms9 male-fertility polynucleotide sequences include (a) a polynucleotide comprising the sequence set forth in SEQ ID NO:16, 18, or 20; (b) a polynucleotide having at least 85%, 90% or 95% sequence identity to SEQ ID NO: 16, 18, or 20; (c) a polynucleotide that encodes a polypeptide having at least 85%, 90% or 95% sequence identity to SEQ ID NO:17, 19, or 21; and (d) a polynucleotide that encodes a polypeptide of SEQ ID NO: 17, 19, or 21.

In some embodiments, the wheat Ms22 male-fertility polynucleotide sequences include (a) a polynucleotide comprising the sequence set forth in SEQ ID NO:22, 24, or 26; (b) a polynucleotide having at least 85%, 90% or 95% sequence identity to SEQ ID NO: 22, 24, or 26; (c) a polynucleotide that encodes a polypeptide having at least 85%, 90% or 95% sequence identity to SEQ ID NO: 23, 25, or 27; and (d) a polynucleotide that encodes a polypeptide of SEQ ID NO: 23, 25, or 27.

In some embodiments, the wheat Ms26 male-fertility polynucleotide sequences include (a) a polynucleotide comprising the sequence set forth in SEQ ID NO:28, 30, or 32; (b) a polynucleotide having at least 85%, 90% or 95% sequence identity to SEQ ID NO: 28, 30, or 32; (c) a polynucleotide that encodes a polypeptide having at least 85%, 90% or 95% sequence identity to SEQ ID NO: 29, 31, or 33; and (d) a polynucleotide that encodes a polypeptide of SEQ ID NO: 29, 31, or 33.

In some embodiments, the wheat Ms45 male-fertility polynucleotide sequences include (a) a polynucleotide comprising the sequence set forth in SEQ ID NO:34, 36, or 38; (b) a polynucleotide having at least 85%, 90% or 95% sequence identity to SEQ ID NO: 34, 36, or 38; (c) a polynucleotide that encodes a polypeptide having at least 85%, 90% or 95% sequence identity to SEQ ID NO: 35, 37, or 39; and (d) a polynucleotide that encodes a polypeptide of SEQ ID NO: 35, 37, or 39.

TABLE 1
Summary of SEQ ID NOS:

| SEQ ID NO: | Description |
|---|---|
| 1 | Wheat Ms1 A genomic (exon- |
| | intron) |
| 2 | Wheat Ms1 A amino acid |
| 3 | Wheat Ms1 B genomic (exon- |
| | intron) |
| 4 | Wheat Ms1 B amino acid |
| 5 | Wheat Ms1 D genomic (exon- |
| | intron) |
| 6 | Wheat Ms1 D amino acid |
| 7 | Wheat Ms5 3A genomic (exon- |
| | intron) |
| 8 | Wheat Ms5 3A amino acid |
| 9 | Wheat Ms5 3B genomic (exon- |
| | intron) |
| 10 | Wheat Ms5 3B amino acid |
| 11 | Wheat Ms5 3B amino acid |
| 12 | Wheat Ms5 3D genomic (exon- |
| | intron) |
| 13 | Wheat Ms5 3D amino acid |
| 14 | Wheat Ms5 3D genomic (exon- |
| | intron) |
| 15 | Wheat Ms5 3D amino acid |
| 16 | Wheat Ms9 4AS genomic |
| | (exon/intron) |
| 17 | Wheat Ms9 4AS amino acid |
| 18 | Wheat Ms9 4BL genomic |
| | (exon/intron) |
| 19 | Wheat Ms9 4BL amino acid |
| 20 | Wheat Ms9 4DL genomic |
| | (exon/intron) |
| 21 | Wheat Ms9 4DL amino acid |
| 22 | Wheat Ms22 AL genomic |
| | (exon/intron) |
| 23 | Wheat Ms22 AL amino acid |
| 24 | Wheat Ms22 BL genomic |
| | (exon/intron) |
| 25 | Wheat Ms22 BL amino acid |
| 26 | Wheat Ms22 DL genomic |
| | (exon/intron) |
| 27 | Wheat Ms22 DL amino acid |
| 28 | Wheat Ms26 4AS genomic |
| | (exon/intron) |
| 29 | Wheat Ms26 4AS amino acid |
| 30 | Wheat Ms26 4BL genomic |
| | (exon/intron) |
| 31 | Wheat Ms26 4BL amino acid |
| 32 | Wheat Ms26 4DL genomic |
| | (exon/intron) |
| 33 | Wheat Ms26 4DL amino acid |
| 34 | Wheat Ms45 4AS genomic |
| | (exon/intron) |
| 35 | Wheat Ms45 4AS amino acid |
| 36 | Wheat Ms45 4BL genomic |
| | (exon/intron) |
| 37 | Wheat Ms45 4BL amino acid |
| 38 | Wheat Ms45 4DL genomic |
| | (exon/intron) |
| 39 | Wheat Ms45 4DL amino acid |
| 40 | Guide for gene editing |
| 41 | Thinopyrum ponticum blue |
| | aleurone amino acid |
| 42 | Thinopyrum ponticum blue |
| | aleurone amino acid |

In some aspects, the plant-derived polynucleotide that confers the plant phenotypic marker and the male-fertility restoration locus are natively linked to each other on the plant restoration donor chromosomal component. In some aspects, the plant-derived polynucleotide that confers the plant phenotypic marker and the male-fertility restoration locus are natively linked to each other and are both located on the same side of the centromere on the plant restoration donor chromosomal component, that is, on the same chromosomal arm. In some embodiments, they are both are linked together and located on the long arm of the plant restoration donor chromosomal component or both located on the short arm of the plant restoration donor chromosomal component and not separated by a centromere. The present disclosure described herein is based, in part, on the discovery that the 4EL chromosome from Blue Norco is able complement Tams45-abd mutations, see, for example, Example 1, herein. In one embodiment, the plant phenotypic marker is blue aleurone and is on the same side of the centromere as a male-fertility restoration locus that functionally complements the male-sterility phenotype in a male-sterile female wheat plant that has homozygous Ms45 mutations.

The plant restoration donor chromosomal component may be from any plant so long as it is able to restore fertility to a male-sterile plant so that the plant produces viable pollen capable of fertilizing a plant, for example, itself. In some aspects, the plant restoration donor chromosomal component is from maize, rice, sorghum, rye, barley, wheat, millet, oats, sugarcane, turfgrass, triticale, switchgrass, wheatgrass, fonio, soybean, canola, alfalfa, sunflower, cotton, tobacco, peanut, potato, tobacco, Arabidopsis, safflower, Thinopyrum, Aegilops, Secale, Haynaldia, Elyymus, Hordeum, or a related species thereof. In some aspects, the plant restoration donor chromosomal component comprises chromosome 4 from a plant. In some embodiments, the plant restoration donor chromosomal component is a 4E, 4EL, or 4H chromosome from a species of Triticum, Thinopyrum, Aegilops, Secale, Haynaldia, Elyymus, or Hordeum, or a related species thereof. The orthologous counterpart of the plant restoration donor chromosomal component in other species may be on the same chromosome, e.g. 4, or located on a different chromosome. In some aspects, the plant phenotypic marker and the male-fertility restoration locus are linked together on chromosome 4 which may be used as a plant restoration donor chromosomal component.

In some cases, the male-fertility restoration locus and the plant-derived polynucleotide that confers the plant phenotypic marker are not closely linked or natively linked. Molecular and biological techniques including genome editing technology such as CRISPR, Talons, meganucleases, and the like, can be used to increase their genetic linkage and/or decrease their physical distance on the plant restoration donor chromosomal component.

In some examples, one or more plant-derived polynucleotides conferring the plant phenotypic marker is inserted in, repositioned within, or rearranged on the plant restoration donor chromosomal component to increase the genetic linkage, decrease the recombination frequency, lower the cross-over frequency, and/or decrease the physical distance or combinations thereof with respect to one or more male-fertility restoration locus on the plant restoration donor chromosomal component. One or more of the plant-derived polynucleotide conferring the plant phenotypic marker may be endogenous or exogenous with respect to the plant restoration donor chromosomal component and/or one or more male-fertility restoration locus. One or more of the male-fertility restoration locus may be endogenous or exogenous with respect to the plant restoration donor chromosomal component.

In some examples, one or more male-fertility restoration locus is inserted in, repositioned within, or rearranged on the plant restoration donor chromosomal component to increase the genetic linkage, decrease the recombination frequency, lower the cross-over frequency, and/or decrease the physical distance or combinations thereof with respect to one or more plant-derived polynucleotides conferring the plant phenotypic marker on the plant restoration donor chromosomal component. One or more of the male-fertility restoration locus may be endogenous or exogenous with respect to the plant restoration donor chromosomal component or the one or more plant-derived polynucleotides conferring the plant phenotypic marker. One or more of the plant-derived polynucleotides conferring the plant phenotypic marker may be endogenous or exogenous with respect to the plant restoration donor chromosomal component.

In other embodiments, the male-fertility restoration locus and the plant-derived polynucleotide conferring the plant phenotypic marker have been edited, inserted, repositioned, or rearranged or combinations thereof so that they are both at different locations on the plant restoration donor chromosomal component than their native locations on the plant restoration donor chromosomal component. In some examples, the plant phenotypic marker and male-fertility restoration locus are located on the same side of the centromere of the plant restoration donor chromosomal component. In some embodiments where there is more than one male-fertility restoration locus and/or plant-derived polynucleotide conferring the plant phenotypic marker, at least one male-fertility restoration locus and at least one plant-derived polynucleotide are at different locations on the plant restoration donor chromosomal component than their native locations. In some embodiments, the plant-derived polynucleotide conferring the plant phenotypic marker and the one or more male-fertility restoration locus have less physical distance between them as compared to the physical distance between the original plant-derived polynucleotide and the original male-fertility restoration locus when located at their native locations. In some embodiments, the plant-derived polynucleotide conferring the plant phenotypic marker and the one or more male-fertility restoration locus have increased genetic linkage between them as compared to the genetic linkage between the original plant-derived polynucleotide and the original male-fertility restoration locus when located at their native locations. In some aspects, the plant-derived polynucleotide conferring the plant phenotypic marker and the one or more male-fertility restoration locus have decreased recombination frequency as compared to the recombination frequency between the original plant-derived polynucleotide and the original male-fertility restoration locus when located at their native locations. In some aspects, the plant-derived polynucleotide conferring the plant phenotypic marker and the one or more male-fertility restoration locus have lower cross-over frequency as compared to the cross-over frequency between the original plant-derived polynucleotide and the original male-fertility restoration locus when located at their native locations.

In some embodiments, where one or more male-fertility restoration locus has been inserted into the plant restoration donor chromosomal component, the native or original male-fertility restoration locus may be disrupted so that it no longer expresses and cannot restore male-fertility in a male-sterile plant. In some aspects, the method may include disrupting the native or original male-fertility restoration locus if it does not reside on the same side of the centromere of the plant restoration donor chromosomal component as the plant-derived polynucleotide conferring the plant phenotypic marker or is not closely linked to the plant-derived polynucleotide from a genetics perspective.

In some embodiments, where one or more plant-derived polynucleotides conferring the plant phenotypic marker has been inserted into the plant restoration donor chromosomal component, the native or original plant-derived polynucleotide conferring the plant phenotypic marker may be disrupted so that it no longer expresses and cannot confer a marker phenotype to a plant or parts thereof. In some aspects, the method may include disrupting the native or original plant-derived polynucleotide conferring the plant phenotypic marker if it does not reside on the same side of the centromere of the plant restoration donor chromosomal component as the male-fertility restoration locus or is not closely linked to the male-fertility restoration locus from a genetics perspective.

In some embodiments, the plant restoration donor chromosomal component has one or more modified plant-derived polynucleotides that confer a plant phenotypic marker linked to one or more male-fertility restoration loci. For example, the modified plant-derived polynucleotides that confer a plant phenotypic marker may be modified in its polynucleotide sequence, copy number, expression level, or location within the plant restoration donor chromosomal component as compared to the polynucleotide sequence, copy number, expression level, or location of a native, non-modified plant-derived polynucleotide that confers a plant phenotypic marker.

In some embodiments, the plant restoration donor chromosomal component has one or more plant-derived polynucleotides that confer a plant phenotypic marker linked to one or more modified male-fertility restoration loci. For example, the modified male-fertility restoration loci may be modified in its polynucleotide sequence, copy number, expression level, or location within the plant restoration donor chromosomal component as compared to the polynucleotide sequence, copy number, expression level, or location of a native, non-modified male-fertility restoration locus.

In some embodiments, the plant restoration donor chromosomal component has one or more modified plant-derived polynucleotides that confer a plant phenotypic marker linked to one or more modified male-fertility restoration loci. For example, the modified male-fertility restoration loci may be modified in its polynucleotide sequence, copy number, expression level, or location within the plant restoration donor chromosomal component as compared to the polynucleotide sequence, copy number, expression level, or location of a native, non-modified male-fertility restoration locus. For example, the modified plant-derived polynucleotides that confer a plant phenotypic marker may be modified in its polynucleotide sequence, copy number, expression level, or location within the plant restoration donor chromosomal component as compared to the polynucleotide sequence, copy number, expression level, or location of a native, non-modified plant-derived polynucleotide that confers a plant phenotypic marker.

In some aspects, the plant-derived polynucleotide that confers the plant phenotypic marker and the male-fertility restoration locus reside on opposite arms of the chromosome. Any suitable technology and technique may be used to bring the plant-derived polynucleotide that confers the plant phenotypic marker and the male-fertility restoration locus to the same arm of the chromosome, for example, translocation, genome editing, pericentric inversions, or combinations thereof.

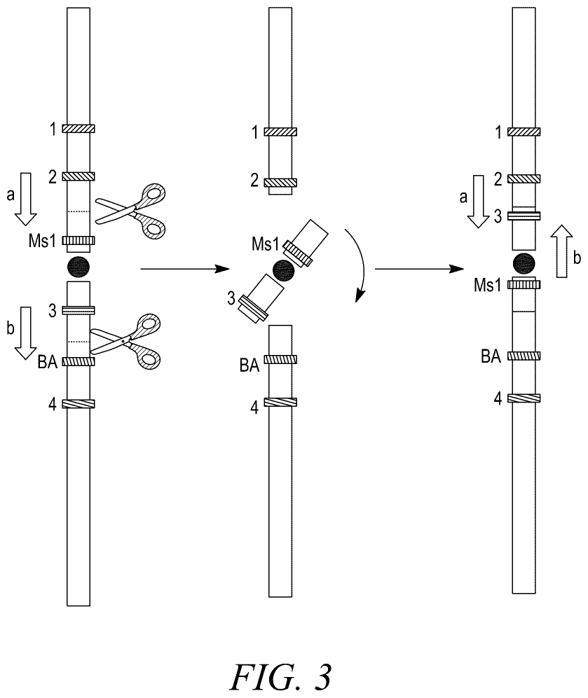

In one embodiment, the Ms1 male-fertility restoration locus and the plant-derived polynucleotide encoding the plant phenotypic marker of blue aleurone (Ba1) gene are initially located on different chromosome arms. In some aspects, the Ms1 male-fertility restoration locus resides on chromosome 4ES from Agropyron elongatum or 4HS from barley (Hordeum vulgare) and the blue aleurone (Ba1) gene on 4EL from Blue Norco. In one embodiment, double-strand breaks in a hybrid (chimeric) plant restoration donor chromosomal component of 4HS-4EL are induced, for example, one double-strand break between the barley Ms1-H gene and the telomere of 4HS, and one double-strand break between the Ba1 gene and the centromere of 4HS-4EL. The chromosome fragment containing the centromere and Ms1-H will be rejoined to the telomeric ends in the opposite orientation, resulting in a pericentric inversion, with Ms1-H and Ba1 now located on the same arm of the new chromosome. See, for example, FIG. 3.

The plant restoration donor chromosomal component may be introduced into a plant cell, plant part, or plant, for example, a male-sterile plant, using any suitable technique known to those in the art. In some approaches, the plant restoration donor chromosomal component is introduced into the plant cell, plant part, or plant using genome editing, transformation, embryo culture, or chromosomal translocation techniques. In some embodiments, the plant cell is a wheat plant cell that has one or more homozygous mutations in a male-fertility polynucleotide, for example, Ms45, and a plant restoration donor chromosomal component that functionally complements the male-sterile homozygous mutations conferring male-sterility to a wheat plant.

The plant restoration donor chromosomal component may be translocated into a plant's genome using any suitable methods. The translocation may be a Robertsonian or non-Robertsonian translocation. In some aspects, the plant restoration donor chromosomal component is translocated and substitutes for an existing plant chromosomal arm, for example, a wheat, barley, or rye plant. In some embodiments, the plant restoration donor chromosomal component may be used to replace the short or long arm of the plant's chromosome, e.g. wheat, barley, or rye.

In some embodiments, the plant restoration donor chromosomal component is introduced using a targeted approach so that the plant restoration donor chromosomal component is introduced into a location in the recipient plant's genome so that any potential undesirable effects on the recipient plant are minimized, avoided or the targeted location provides potential beneficial advantages. For example, the plant restoration donor chromosomal component may be introduced or substituted for a chromosomal arm in a wheat plant where the wheat plant lacking that chromosomal arm or pair of chromosomal arms (monosomic or ditelosomoic) has a normal phenotype. In another example, the plant restoration donor chromosomal component may be introduced or substituted for a chromosomal arm in a wheat plant where the lack of the chromosomal arm would have the effect of reducing transmission rates of the plant restoration donor chromosomal component, for example, reducing male or female transmission rates. In one embodiment, the plant restoration donor chromosomal component is substituted for the long arm of chromosome 5A (5AL) in a wheat plant, so that the result is the plant restoration donor chromosomal component-5AS.

In some examples, having a translocated and/or substituted plant restoration donor chromosomal component may confer advantages over an independent (addition) plant restoration donor chromosomal component. One advantage is reduced gametic transmission of the independent (addition) plant restoration donor chromosomal component, which allows for an increased percentage of seed that contain the one or more homozygous mutations in the male-fertility polynucleotide and do not contain the plant restoration donor chromosomal component to be produced during female seed increase.

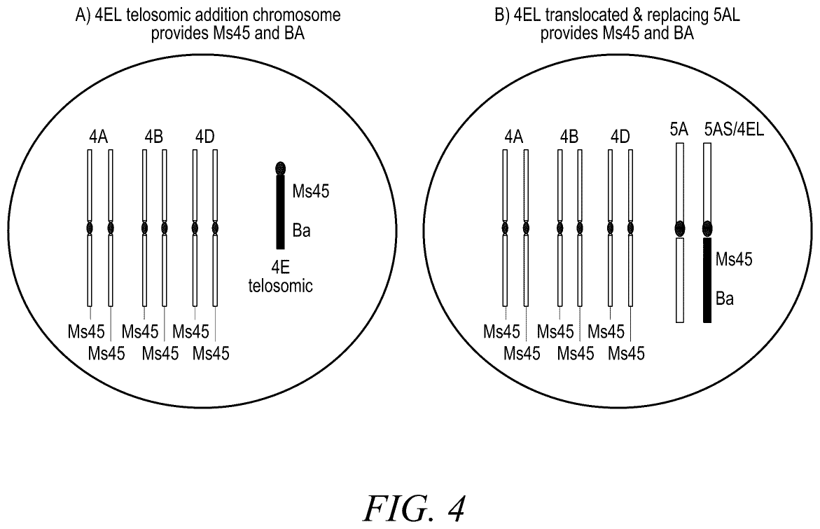

In some embodiments, a translocated, substituted plant restoration donor chromosomal component may be created via a Robertsonian translocation. See, for example, FIG. 4. Aneuploid stocks may be used to create wheat-chromosomal donor Robertsonian translocations in a directed manner by making the appropriate wheat and donor chromosomes in monosomic condition. The translocated, substituted plant restoration donor chromosomal component may be introgressed along with one or more homozygous mutations in the male-fertility polynucleotide or a single fertility gene recessive into various elite female lines to facilitate hybrid seed production. Since Ms1 behaves as a single gene recessive, in some embodiments, only the one mutation in the Ms1 polynucleotide or allele located on chromosome 4BS may need to be introgressed into female wheat lines to produce male-sterility.

In one example, the plant restoration donor chromosomal component is introduced into the male-sterile plant using breeding techniques. In some examples, the male-sterile plant is crossed with a plant comprising the plant restoration donor chromosomal component. The plant restoration donor chromosomal component may be introduced into a plant, for example, a male-sterile plant, using a plant that has the same base number of chromosomes as the recipient (e.g. male-sterile) plant. In some examples, the male-sterile plant is a wheat plant, e.g. diploid, tetraploid, or hexaploid. In some examples, the plant restoration donor chromosomal component may be from or introduced from a wheat plant, e.g. diploid, tetraploid, or hexaploid wheat, an Aegilops, Secale, Agropyrin, Haynaldia, Hordeum, or Elyymus plant, or any other plant that the male-sterile plant is cross-compatible with and can be fertilized by.

In some aspects, the plant restoration donor chromosomal component may be exogenous with respect to the recipient plant, e.g. a male-sterile plant, or its host plant, e.g. a male-fertile plant. For example, the plant restoration donor chromosomal component may be from a plant that is of a different species or plant than the male-sterile plant, for example, having a plant restoration donor chromosomal component from wheatgrass or barley in a wheat plant. Accordingly, in some examples, the plant restoration donor chromosomal component is from a non-wheat plant or species. In some embodiments, the plant restoration donor chromosomal component does not pair or recombine with any of the male-sterile female plant's chromosomes, e.g. wheat chromosomes.

The plant restoration donor chromosomal component may be from any wild or cultivated plant including but not limited to maize, rice, sorghum, rye, barley, wheat, millet, oats, sugarcane, turfgrass, triticale, switchgrass, wheatgrass, Thinopyrum, Aegilops, Secale, Haynaldia, Elyymus, Hordeum, soybean, canola, alfalfa, sunflower, cotton, maize, or a related species thereof. The plant restoration donor chromosomal component may be from any number of plant species, including but not limited to Thinopyrum, Aegilops, Secale, Haynaldia, Elyymus, or Hordeum species. In some embodiments, the plant restoration donor chromosomal component may be from a wheat line having one or more chromosomes from maize, rice, sorghum, rye, barley, wheat, millet, oats, sugarcane, turfgrass, triticale, switchgrass, wheatgrass, Thinopyrum, Aegilops, Secale, Haynaldia, Elyymus, or Hordeum, soybean, canola, alfalfa, sunflower, cotton, maize, or a related species thereof. The plant restoration donor chromosomal component may be introduced and/or from a telosomic addition line, such as a monotelosomic or ditelosomic addition line, or a disomic addition line. In some examples, the plant having the plant restoration donor chromosomal component is a from a wheat line, including but not limited to a Blue Sando, Blue Baart, Blue Onas, Blue 1, PBB, or Blue Norco wheat line. Non-limiting examples include but are not limited to the wheat variety Blue Baart, which has a disomic addition of Thinopyron ponticum chromosome 4E, Blue Norco, which has ditelosomic addition of Thinopyron ponticum chromosome 4E, or a wheat line which has a disomic addition of Hordeum vulgare (barley) chromosome 4H. Blue Norco, aneuploid, monosomic, disomic lines, and other wheat lines are publicly available and may be obtained from a number of centers, such as the National Small Grains Collection (United States Department of Agriculture-Agricultural Research Service, National Small Grains Collection, Aberdeen, Id. 83210 USA), or Wheat Genetics Resource Center (Kansas State University, Kansas, USA).

In some embodiments, the plant restoration donor chromosomal component is from two, three, or more of the same or different species to make a hybrid (chimeric) plant restoration donor chromosomal component. In some embodiments, the hybrid (chimeric) plant restoration donor chromosomal component has the plant-derived polynucleotide that confers the plant phenotypic marker from one plant or species and the male-fertility restoration locus from a different plant or species. The plant-derived polynucleotide that confers the plant phenotypic marker in the hybrid (chimeric) plant restoration donor chromosomal component may have one or more chromosomes or chromosomal fragments from maize, rice, sorghum, rye, barley, wheat, millet, oats, sugarcane, turfgrass, triticale, switchgrass, wheatgrass, Thinopyrum, Aegilops, Secale, Haynaldia, Elyymus, Hordeum, soybean, canola, alfalfa, sunflower, cotton, maize, or a related species thereof. The male-fertility restoration locus in the hybrid (chimeric) plant restoration donor chromosomal component may have one or more chromosomes or chromosomal fragments from maize, rice, sorghum, rye, barley, wheat, millet, oats, sugarcane, turfgrass, triticale, switchgrass, wheatgrass, Thinopyrum, Aegilops, Secale, Haynaldia, Elyymus, Hordeum, soybean, canola, alfalfa, sunflower, cotton, maize, or a related species thereof. In one example, the plant phenotypic marker is from Thinopyrum ponticum, for example, chromosome 4E, and the fertility restoration locus from Hordeum vulgare (barley), for example, chromosome 4H, to make a hybrid (chimeric) plant restoration donor chromosomal component of 4H-4E. In some embodiments, the hybrid (chimeric) plant restoration donor chromosomal component includes the short arm of chromosome 4 from Hordeum vulgare (barley) and the long arm of chromosome 4E from Thinopyron ponticum, for example, from Blue Norco to make a hybrid (chimeric) plant restoration donor chromosomal component of 4HS-4EL. The plant phenotypic marker and fertility restoration locus may be from any suitable lines, including but not limited to disomic additions, such as barley 4H, Thinopyrum ponticum chromosome 4E, Agropyron 4E, or combinations thereof.

Confirmation may be made that the plant, plant part, plant cell, or seed contains the plant restoration donor chromosomal component, including o the phenotypic marker or male-fertility restoration locus or both using routine and well-known methods. Plant restoration donor chromosomal components should retain male-fertility restoration activity in plants, particularly the ability to promote male tissue development. The plant's male-fertility condition can be assessed by any suitable technique, for example, by observation of the plant's male tissue development, such as phenotyping of anthers and seed set on individual plants. See, for example, Example 1 herein.

Absence of the plant restoration donor chromosomal component, for example, lost by crossing or mutation, may be evidenced by plants lacking the male-fertility phenotype or by plants, plant parts, plant cells, or seeds lacking the phenotype conferred by the plant-derived phenotypic marker as compared to a control. Alternatively, or additionally, confirmation of the presence or absence of the plant restoration donor chromosomal component may occur later, for example, after multiple plant crossings or from subsequent generations.

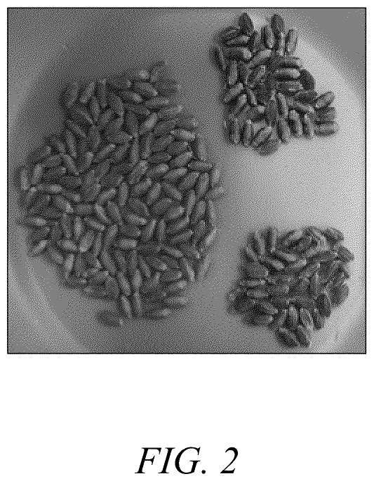

Seeds produced from the plants or crosses may be harvested together and sorted into separate populations if desired. For example, seeds comprising the plant-derived plant phenotypic seed marker linked to the fertility restoration locus are seeds that may be sorted and separated for use as a maintainer and the seeds lacking the plant-derived plant phenotypic marker linked to the fertility restoration locus (plant restoration donor chromosomal component) may be grown and used as male-sterile female parents in hybrid crosses. The seeds may be manually, mechanically, or optically sorted into these populations. To facilitate high throughput and analysis, the sorting may employ a semi-automated or automated approach. Populations of seeds may be sorted using optical sensing technology including multi or hyper spectral imaging, uv, visible or NIR spectroscopy systems, and/or optical scanning. For example, when the plant phenotypic marker from the plant restoration donor chromosomal component is a color marker such as aleurone, the homozygous blue seed will be more intensely colored than heterozygous blue seed and may sorted on that basis, e.g. the seeds having different concentrations or expression levels of the blue aleurone. See, for example, FIG. 2. Additionally or alternatively, seeds may be evaluated for the presence of the plant restoration donor chromosomal component, for example, the plant phenotypic marker, using any other suitable technique, including but not limited to flow cytometry or qPCR.

Alternately, the seed may be mixed (unsorted) so that a first portion of seeds contain the one or more homozygous mutations of the male-fertility polynucleotide (seeds that will give rise to male-sterile female plants) and a second portion of seeds contain the one or more homozygous mutations of the male-fertility polynucleotide and the plant restoration donor chromosomal component comprising a plant-derived polynucleotide that confers the plant phenotypic marker linked to the male-fertility restoration locus (seeds that will give rise to male-fertile plants). A plant phenotypic marker that is a seed color marker may be used to sort and separate hybrid wheat or inbred wheat seeds from maintainer wheat seeds. In some examples, the mixture of seeds are planted together to increase the number of male-sterile female seeds produced. The mixture of seeds may be placed in a bag or other appropriate container. The method may include planting the mixture of seeds of male and female parent plants in the same row in a field, rather than in separate rows. The male and female parent plants are grown and the male parent plants fertilize the female parent plants to produce seed. The resulting seed will be a mixture of seeds that, when planted, will give rise to male-sterile or male-fertile plants. The In some examples, the percentage of seeds produced that will give rise to male-fertile plants may be at least about 20%, 25%, 30%, 35%, 40% or 45% of the seeds produced. In some examples, the percentage of seeds produced that will give rise to male-sterile female plants is at least about 55%, 60%, 65%, 70%, 75%, 80%, 85% or 90% of the seeds produced. In some aspects, this method increases the number of seeds produced that will give rise to male-sterile female plants as compared the number of male-sterile female seeds produced in a field where the female-sterile seeds and male-sterile seeds are not planted together in the some row, but are each planted in separate rows of male and female rows.

Also provided herein is a plant cell, plant, or seed obtained or produced from the methods described herein. In some methods, a male-sterile female plant is fertilized with pollen from a plant comprising the plant restoration donor chromosomal component that complements the female plant's male-sterile condition. This method produces seed that may be a mixture of seed that, when planted, gives rise to male-sterile or male-fertile plants. The absence of the plant phenotypic marker in the seeds indicates that the seed lacks the plant restoration donor chromosomal component and, when planted, will give rise to male-sterile female plants. Plants from these seeds may be used as male-sterile female inbreds for hybrid seed production. The presence of the plant phenotypic marker in the seed indicates that the seed contains the plant restoration donor chromosomal component and will give rise to male-fertile plants. Plants grown from this seed may be used as a maintainer if desired. The seed may be unsorted, mixed with other seed, or sorted and separated into populations using routine techniques and instruments as described elsewhere herein and known to one in the art.

Since a male sterile plant, e.g a male sterile wheat plant, cannot be maintained by itself, provided herein are compositions and methods for maintaining the homozygous recessive male sterility condition of the wheat plant that include the use of a plant restoration donor chromosomal component for restoration of male fertility to the wheat plant. For example, a mutation in a gene critical to male fertility can impart a male sterility phenotype to wheat plants when this mutant allele is in the homozygous state, for example, in endogenous wheat Ms1, Ms5, Ms9, Ms22, Ms26, and Ms45 polynucleotides encoding wheat Ms1, Ms5, Ms9, Ms22, Ms26, and Ms45 polypeptides respectively. Since Ms1 in wheat behaves as a single gene recessive, in some embodiments only the ms1 male-fertility polynucleotide or allele located on chromosome 4BS may need to be mutated to confer male-sterility to a wheat plant.

When a male-fertility restoration locus that is capable of functionally complementing homozygous recessive alleles that confer male sterility is introduced into and expressed in the male-sterile plant, male-fertility is restored to the plant so that it can produce viable pollen and is capable of fertilizing a cross-compatible female plant.

Maintenance of the homozygous recessive condition or male-sterility condition may include introducing into the wheat plant a plant restoration donor chromosomal component to create a maintainer plant. The plant restoration donor chromosomal component, upon introduction into a plant that has one or more homozygous mutations of a male-fertility polynucleotide, restores the male-fertility to the plant so that the plant produces viable pollen capable of fertilizing itself or a cross-compatible plant. In some embodiments, the plant restoration donor chromosomal component may be present in the maintainer wheat plant as an additional chromosome in the wheat genome, as a translocated chromosome in the wheat genome, or as a substitution chromosome for a wheat chromosome in the wheat genome. Accordingly, provided herein is a wheat plant or cell that has one or more of its wheat chromosomes substituted by a plant restoration donor chromosomal component from a non-wheat plant or species. For example, in some embodiments, the wheat plant or cell has a homoeologous chromosome pair of two chromosomes. The first chromosome is native to the wheat plant and the second chromosome comprises a plant restoration donor chromosomal component. The plant restoration donor chromosomal component may be translocated with or substituted for any wheat chromosomes so long as it does not have a deleterious effect on the wheat plant. In some embodiments, the addition, translocation, or substitution should not interfere with the one or more wheat plant's homozygous mutations that confer the male-sterile phenotype. In some embodiments, the plant restoration donor chromosomal component may be substituted for or translocated into a 5AL wheat chromosome.

Wheat seeds from these plants having chromosomal substitutions are euploid but have one or more of chromosomes substituted by or contain the plant restoration donor chromosomal component. Confirmation of the number of chromosomes in the wheat plant or cell or the plant restoration donor chromosomal component's translocation or substitution may be detected using any appropriate technique, such as genomic in situ hybridization (GISH) or fluorescent in situ hybridization (FISH).

The male-sterility condition of the wheat plant may be maintained by self-fertilizing the maintainer plant comprising the plant restoration donor chromosomal component which will result in the creation of a mixed seed population. A portion of the seed will contain the one or more homozygous male-sterility mutations and a portion of the seed will contain the one or more homozygous male-sterility mutations and the plant restoration donor chromosomal component. The seed resulting from self-fertilization of the maintainer can be planted and the seed from these plants selected and sorted for use in male-sterility maintenance or hybrid seed production. In some examples, the selection process utilizes any suitable seed marker as a plant phenotypic marker to identify such seed. Accordingly, provided herein are methods for producing seed from a wheat plant by self-fertilizing the maintainer plant. In other embodiments, a male-sterile wheat plant homozygous for one or more mutations of a male-fertility polynucleotide may be fertilized by pollen from a male-fertile plant that it is cross-compatible with. In some aspects, the male-fertile plant contains a plant restoration donor chromosomal component.

Also included herein are methods and compositions for restoring male fertility to a plant having one or more homozygous recessive mutations in a male fertility polynucleotide that confers male sterility to the plant. In some aspects, the method includes introducing the plant restoration donor chromosomal component that functionally complements the male-sterility phenotype from the one or more homozygous mutations in the male-sterile wheat plant so that the wheat plant becomes male-fertile. The plant restoration donor chromosomal component may be introduced using any number of approaches as described herein and known to one skilled in the art.

The plants or plant restoration chromosomal component for use in the methods and compositions described herein may be a monocot or a dicot. Monocots include but are not limited to maize, rice, sorghum, rye, barley, wheat, millet, oats, triticale, fonio, sugarcane, turfgrass, switchgrass, Thinopyrum, Aegilops, Secale, Haynaldia, Elyymus, or Hordeum, or a related species thereof. Dicots include but not limited to soybean, canola, alfalfa, sunflower, cotton, tobacco, peanut, potato, tobacco, Arabidopsis, or safflower, or a related species thereof.

### Additional Definitions

As used herein and in the appended claims, the singular forms “a”, “an”, and “the” include plural reference unless the context clearly dictates otherwise. Thus, for example, reference to “a plant” includes a plurality of such plants; reference to “a cell” includes one or more cells and equivalents thereof known to those skilled in the art, and so forth.

The term “crossed” or “cross” or “crossing” in the context of this disclosure means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

“Expression” generally refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Gamete” refers to a reproductive cell having the 1n set (haploid number) of chromosomes that can fuse with another gamete of the opposite sex during fertilization in organisms undergoing sexual reproduction. As used herein, a gamete in organisms undergoing asexual reproduction refers to a cell having a 2n number (an unreduced number) of chromosomes.

The term “genome” refers to the entire complement of genetic material (genes and non-coding sequences) that is present in each cell of an organism, or virus or organelle; and/or a complete set of chromosomes inherited as a (haploid) unit from one parent.

As used herein, a “male sterile plant” is a plant that does not produce male gametes that are viable or otherwise capable of fertilization and is female fertile.

The term “plant” refers to whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. Plant parts include differentiated and undifferentiated tissues including, but not limited to roots, stems, shoots, leaves, pollen, seeds, tumor tissue and various forms of cells and culture (e.g., single cells, protoplasts, embryos, and callus tissue). The plant tissue may be in plant or in a plant organ, tissue or cell culture. The term “plant organ” refers to plant tissue or a group of tissues that constitute a morphologically and functionally distinct part of a plant.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

“Polypeptide”, “peptide”, “amino acid sequence” and “protein” are used interchangeably herein to refer to a polymer of amino acid residues.

“Progeny” comprises any subsequent generation of a plant.

“Promoter functional in a plant” is a promoter capable of controlling transcription in plant cells whether or not its origin is from a plant cell.

As used herein “promoter” includes reference to a region of DNA upstream from the start of transcription and involved in recognition and binding of RNA polymerase and other proteins to initiate transcription.

A “plant promoter” is a promoter capable of initiating transcription in plant cells.

The terms “suppress”, “suppressed”, “suppression”, “suppressing” and “silencing”, are used interchangeably herein and include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches and the like.

As used herein, the term “wheat” refers to any species of the genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. mocha, T. compactum, T. sphaerococcum, T. vavilovii, and interspecies cross thereof. Tetraploid wheat includes T. durum (also referred to as durum wheat or Triticum turgidum ssp. durum), T. dicoccoides, T. dicoccum, T. polonicum, and interspecies cross thereof. In addition, the term “wheat” includes possible progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. A wheat cultivar for use in the present disclosure may belong to, but is not limited to, any of the above-listed species. Also encompassed are plants that are produced by conventional techniques using Triticum sp. as a parent in a sexual cross with a non-Triticum species, such as rye (Secale cereale), including but not limited to Triticale. In some aspects, the wheat plant is suitable for commercial production of grain, such as commercial varieties of hexaploid wheat or durum wheat, having suitable agronomic characteristics which are known to those skilled in the art.

### EXAMPLES

In the following Examples, unless otherwise stated, parts and percentages are by weight and degrees are Celsius. It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims.

### EXAMPLE 1: A Mono-Telosomic Chromosome From Thinopyrum ponticum can Complement Ms45 Triple Mutations and Restore Fertility

As demonstrated herein, the 4EL chromosome from Blue Norco is able complement Tams45-abd mutations. Blue Norco, a blue aleurone wheat, is a ditelosomic addition line which has an extra set of 4EL (2 n=42″+2t) that is available from USDA-ARS (PI 542465). Tams45-abd plants were crossed to Blue Norco as a male parent. The crossed seeds (blue in color) were planted, and F1 progeny screened to confirm the presence TaMS45-A, -B and -D mutations and allowed to self-pollinate.

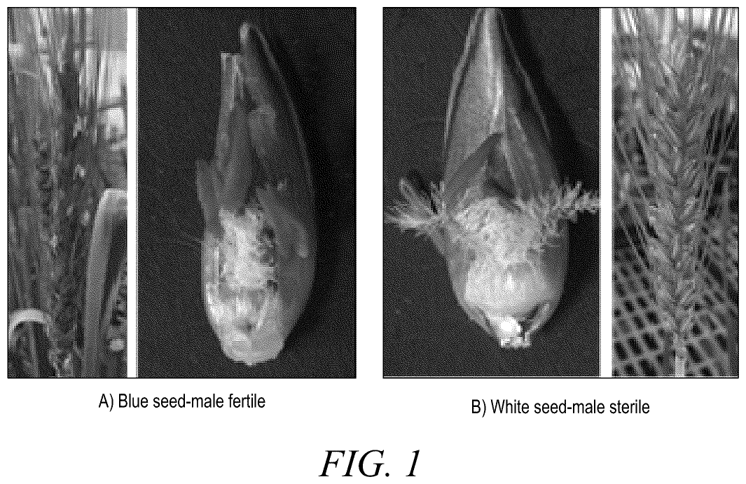

Light blue F2 seed (monosomic for 4EL, i.e. one copy of 4EL) were planted and progeny were genotyped for TaMs45 mutant alleles and allowed to self-pollinate and set seed. Of the harvested seeds from these plants, 64 light blue seeds (with 4EL) and white seeds (without 4EL) each were planted to obtain F3 plants which were genotyped for TaMs45 mutant alleles. Triple homozygous (Tams45-abd) mutant plants from both white and blue kernels were identified and analyzed for male fertility through phenotyping of anthers and seed set on individual plants. In total, 12 triple homozygous Tams45-abd plants were analyzed, six from white seeds and six from blue seeds (Table 2). The anthers of the Tams45-abd plants from white seeds were shriveled in shape and smaller in size, whereas the anthers of Tams45-abd plants from blue kernels were similar to wild-type plants (FIG. 1). All six Tams45-abd plants that carried 4EL chromosome were male-fertile with seed set comparable to wild-type plants while the six plants without the 4EL chromosome showed negligible seed-set (Table 2; Note: seed observed in these plants was likely to due to open fertilization as these heads were not bagged and these plants were in very close proximity with fertile flowering plants).

To test a working male-sterile maintainer system based on blue white selection, seeds from fertile F3 plants were harvested and 12 visually sorted white and light blue seeds from three F3 plants (total=36 white and blue seeds each) were planted to obtain F4 generation. All the 36 plants from the white seeds were male-sterile while all the 36 plants from blue seeds were male-fertile (Table 2). This data demonstrates that a monosomic 4EL chromosome can complement and restore male-fertility and maintain the triple homozygous Tams45 mutants. Further it is possible to identify from the seeds of a maintainer plant, based on the color, the seed which will produce a male-sterile or a male-fertile plant.

TABLE 2
Complementation of TaMs45 triple homozygous mutants with
4EL chromosome.

| Genotype of | Seed Set-Fertility | | | Male | Male |
|---|---|---|---|---|---|
| TaMs45 | | Total | Seed per | fertile | sterile |
| homeologs¹ | Plants | Seed | Plant | plants | plants |
| F3 generation | | | | | |
| Tams45-abd + | 6 | 1635 | 272 | 6 | 0 |
| 4EL | | | | | |
| Tams45-abd | 6 | 68* | 11 | 0 | 6 |
| F4 generation | | | | | |
| Tams45-abd + | 36 | 4858 | 135 | 36 | 0 |
| 4EL | | | | | |
| Tams45-abd | 36 | 0 | 0 | 0 | 36 |
| TaMs45-ABD | 5 | 855 | 171 | 5 | 0 |

*Note:
heads were not bagged and grew in close proximity of same stage fertile plants

### EXAMPLE 2: Production of a 4HS-4EL Hybrid Chromosome

In this example, a 4HS-4EL hybrid chromosome is created using a Robertsonian translocation. For example, aneuploid stocks may be used to create Robertsonian translocations in a directed manner by making the appropriate plant restoration donor chromosomal component in monosomic condition. A Chinese Spring wheat line which has a disomic addition (2n=44) of chromosome 4H from Betzes barley was used in this example. (Wheat stock TA #3700, which has a disomic addition (2n=44) of Hordeum vulgare (barley) chromosome 4H, was obtained from the Wheat Genetics Resource Center at Kansas State University, USA.) The wheat variety Blue Baart, which has a disomic addition (2n=44) of Thinopyrum ponticum chromosome 4E and has blue aleurone was used in this example. (Burešová V, Kopecký D, Bartoš J, Martinek P, Watanabe N, Vyhnánek T and Doležal J. 2015. Variation in genome composition of blue-aleurone wheat. Theor. Appl. Genet. 128:273-282) A cross was made between these two lines, and the resulting F₁ seeds were grown and the plants allowed to self-pollinate. The presence of a new 4HS-4EL chromosome in the F₂ or F₃ generations may be screened using any suitable method, for example, for the presence of blue color, indicative of the presence of 4EL, the absence of markers for 4ES, the presence of a 4HS marker such as Mmag 053, and the absence of a 4HL marker such as HVM40 (Molnár I, Linc G, Dulai S, Nagy E D and Molnár-Láng M M. 2007. Ability of chromosome 4H to compensate for 4D in response to drought stress in a newly developed and identified wheat-barley 4H(4D) disomic substitution line. Plant Breeding 126:369-374). Candidate lines may be further screened, for example, by using standard cytogenetic techniques such as GISH (genomic in situ hybridization) to confirm the presence of a hybrid 4HS-4EL chromosome.

### EXAMPLE 3: Using a CRISPR-cas9-Induced Pericentric Inversion to Place Ms1 and Ba1 on the Same Chromosome Arm

In this example, pericentric inversions are used in wheat to place the Ms1 and Ba1 genes on the same chromosome arm in those instances where the Ms1 complement (homeologue) and the Ba1 gene are on different chromosome arms, for example, located on 4ES or 4HS and 4EL, respectively. One advantage of this approach is that it would place the Ms1 and Ba1 trait genes on the same chromosome arm and avoid their separation due to a breakage of the chromosome at the centromere and possible misclassification of seeds as male-sterile when in fact they are male-fertile.

In this example, the starting material may use the 4HS-4EL chromosome described in Example 2 herein. Two DSBs will be induced: one between the Ms1-H gene and the telomere of 4HS, and one between the Ba1 gene and the centromere of 4HS-4EL. The barley Ms1-H gene is oriented with the 5′ end closest to the telomere and the 3′ end closest to the centromere. The DNA sequence 5′ of the Ms1-H gene (http://plants.ensembl.org/Hordeum_vulgare/Tools/Blast) will be analyzed for the NGG sequence of the Protospacer Adjacent Motif (PAM) for cas9. For each site found, the 17bp upstream sequence will be evaluated to find a unique cutting site which is not present in the wheat genome.

Marker fragments located on the long arm of chromosome 4J (4E^{b}) and located between the centromere and the BaThb gene will be cloned and sequenced; the resulting sequences will be analyzed for the NGG sequence of the Protospacer Adjacent Motif (PAM) for cas9. For each site, the 17bp upstream sequence will be evaluated to find a unique cutting site which is not present in the wheat genome.

When suitable site(s) and guide RNAs for the two locations have been identified, the two DSBs will be induced by CRISPR-cas9 using standard methods. At a certain frequency, the chromosome fragment containing the centromere and Ms1-H will be rejoined to the telomeric ends in the opposite orientation, resulting in a pericentric inversion, with Ms1-H and Ba1 now located on the same arm of the new chromosome. PCR primers will be designed that produce a PCR product only when the pericentric inversion has occurred.

### EXAMPLE 4: Alternative Construction for a 4E-ms45 System

This example describes one embodiment of constructing a hybridization system using 4E-ms45. Rather than the seed color marker (blue aleurone=BA) and functional dominant Ms45 allele being provided by an addition telocentric chromosome such as 4EL, the 4EL chromosome arm will be translocated into the wheat genome, substituting for an existing wheat chromosome arm.

The proper choice of wheat chromosome arm targeted for substitution by 4EL may confer some advantages over an independent (addition) 4EL. One possible advantage is the reduced gametic transmission of the addition telosomic chromosome, for example, a reduced male and/or female transmission rate. This will allow for an increased percentage of non-blue seed to be produced during female seed increase (self-pollination). In this example, 4EL will be substituted for wheat chromosome arm 5AL. Gametes containing a 5AS-4EL chromosome will lack a copy of 5AL. See, for example, FIG. 4.

Even in the case of reduced male and/or female transmission rates, some homozygous 4EL (blue) seed may be produced. The presence of these homozygous blue seeds may be identified and detected using seed sorting. For example, the homozygous blue seed will be more intensely colored than heterozygous blue seed and may be sorted on that basis. It is believed that a 5AS-4EL homozygous plant will likely be male-sterile, so little or no seed sorting would be needed to remove such seeds from the population of blue seeds.

A 5AS-4EL chromosome may be created using a Robertsonian translocation. In this example, aneuploid stocks may be used to create wheat-plant restoration donor chromosomal component Robertsonian translocations in a directed manner by making the appropriate wheat and plant restoration donor chromosomal components in monosomic condition. A Chinese Spring aneuploid wheat line which segregates for monosomy of wheat chromosome 5A was used in this example. (Stock TA #3045, which segregates for monosomy of wheat chromosome 5A, was obtained from the Wheat Genetics Resource Center at Kansas State University, USA.) The wheat variety Blue Baart, which has a disomic addition (2n=44) of Thinopyron ponticum chromosome 4E and has blue aleurone was used in this example (Burešová V, Kopecký D, Bartoš J, Martinek P, Watanabe N, Vyhnánek T and Doležal J. 2015. Variation in genome composition of blue-aleurone wheat. Theor. Appl. Genet. 128:273-282). 10 seeds of the Chinese Spring aneuploid wheat line which segregates for monosomy of wheat chromosome 5A were grown, root tips sampled and chromosome counts were done by standard methods to identify monosomic (2n−1=41) plants. Monosomic plants produce ˜75% nullisomic (n−1=20) female gametes. The identified monosomic plants were crossed as females with Blue Baart. F₁ seeds produced from nullisomic gametes will have the composition of 42 chromosomes, including 1 5A+1 4E, both derived from the male parent Blue Baart and have blue aleurone color. F₁ seeds were grown and the plants allowed to self-pollinate.

The presence of a new 5AS-4EL chromosome may be screened by any suitable method, for example, by the presence of blue aleurone seed color, indicative of the presence of 4EL, and the absence of markers for 4ES. This would be indicative of breakage of the 4E chromosome. Candidate lines may be further screened, for example, by using standard cytogenetic techniques such as FISH (fluorescence in situ hybridization) to confirm the presence of a hybrid 5AS/4EL chromosome.

Once established, such lines will be self-pollinated to confirm male-sterility of 5AS/4EL homozygotes as well as reciprocally crossed as male and female to determine pollen and egg transmission frequencies. The 5AS/4E chromosome may be introgressed along with recessive ms45 alleles on 4A, 4B and 4D into various elite female lines to facilitate hybrid seed production.

### EXAMPLE 5: Use of 4E or 4H Addition Chromosomes to Complement Wheat Ms9 Triple Mutations and to Restore Fertility

In this example, a wheat CAS9-CRISPR construct has been made targeting the TA-Ms9 gene using a guide, TA-Ms9-CR2 ggaggtacaccaactacctg (SEQ ID NO:40).

Wheat plants will be transformed with this construct and the endogenous wheat Ms9 genes will be assessed for mutations in all three wheat genomes. Crosses and subsequent self-pollinations will be made to combine the Ms9 mutations as homozygous into a single plant. Wheat plants homozygous for Ms9 mutations in all three genomes will be evaluated for male sterility through examination of anthers and seed set on individual plants.

If male sterility is achieved, these wheat plants may be maintained by crossing them with a wheat maintainer line containing a 4EL addition chromosome such as Blue Norco, described in Example 1. The TA-Ms9 gene maps to the long arm of chromosome 4 in wheat which has synteny with the long arm of the 4EL addition chromosome, which should provide a complement function to the TA-ms9 mutation, restoring male fertility. A wheat line containing the barley chromosome 4 as an addition chromosome (Hordeum vulgare 4H) may also be evaluated for possible Ta-ms9 restoration. Restoration of male fertility to these male-sterile wheat plants by the 4E, 4EL or 4H addition chromosomes in the wheat maintainer line will be evaluated by the ability to complement the ms9 mutations in these plants, as determined by anther examination and seed set on individual plants.

As described in Example 1, the 4EL addition chromosome also contains the blue aleurone gene (BA) on the long arm, which gives a dominant blue seed phenotype. Therefore, seed from self-pollinated homozygous TA-ms9 mutant and restored with the presence of the 4EL addition chromosome, will segregate for blue and non-blue seed. These resultant seeds can be grown and evaluated for male fertility and sterility. As demonstrated in Example 1, the expectation is that non-blue seed derived plants will be male-sterile and blue seed derived plants will be male fertile, and the latter can act as a maintainer line for the TA-ms9 mutation in a hybrid system.

1.-12. (canceled)

13. A wheat plant cell, the wheat plant cell comprising a homozygous mutation in a Ms45 male-fertility polynucleotide in the A, B, and D wheat genomes that cause a male sterility phenotype and a plant restoration donor chromosomal component comprising a 4E chromosomal component from Thinopyrum or Agropyron, the 4E chromosomal component comprising: a plant polynucleotide that confers a plant seed phenotype linked to a Ms45 male-fertility restoration locus that functionally complements the male-sterility phenotype from the homozygous Ms45 mutations when expressed in a Ms45 male-sterile wheat plant.

14. The wheat plant cell of claim 13, wherein the plant polynucleotide that confers the plant seed phenotype is located on the same chromosomal arm of the 4E chromosomal component as the Ms45 male-fertility restoration locus and not separated by a centromere.

15. The wheat plant cell of claim 13, wherein the plant polynucleotide that confers the plant seed phenotype is a phenotype for color, physiology, or morphology of the seed.

16. The wheat plant cell of claim 15, wherein the color is blue aleurone, P gene, anthocyanin, or Kala 4.

17. The wheat plant cell of claim 13, wherein the plant restoration donor chromosomal component is from a non-wheat species.

18. The wheat plant cell of claim 13, wherein the plant restoration donor chromosomal component is from a Sebesta Blue, Blue Sando, Blue Baart, Blue Onas, Blue 1, PBB, or Blue Norco wheat line comprising the plant restoration donor chromosomal component, wherein the plant restoration donor chromosomal component is derived from a non-wheat species.

19. (canceled)

20. (canceled)

21. A wheat plant obtained from the wheat plant cell of claim 13.

22. Wheat seed from the wheat plant of claim 21, wherein the wheat seed expressing the plant seed phenotype indicate the seed will, when planted, give rise to a male-fertile wheat plant.

23. Wheat seed obtained from selfing the wheat plant of claim 21, wherein the wheat seed comprising the homozygous mutations in the Ms45 male-fertility polynucleotide in the A, B, and D wheat genomes and not comprising the plant polynucleotide that confers the plant seed phenotype indicate the seed will, when planted, will give rise to a male-sterile female wheat plant, wherein the wheat seed comprising the homozygous mutation in the Ms45 male-fertility polynucleotide in the A, B, and D wheat genomes and the plant polynucleotide that confers the plant seed phenotype indicate the seed will, when planted, give rise to a male-fertile wheat plant.

24.-29. (canceled)

30. A method of producing wheat seeds that produce male-sterile female wheat plants, the method comprising:
a. planting a mixture of wheat seeds in a field, wherein the mixture comprises a first portion of wheat seeds comprising a homozygous mutation in a Ms45 male-fertility polynucleotide in the A, B, and D wheat genomes that confer male sterility to a wheat plant, and a second portion of wheat seeds comprising a plant restoration donor chromosomal component comprising a 4E chromosomal component from Thinopyrum or Agropyron, the 4E chromosomal component comprising a plant polynucleotide that confers a plant seed phenotype linked to a Ms45 male-fertility restoration locus, wherein the Ms45 male-fertility restoration locus functionally complements the male-sterility phenotype from the homozygous Ms45 mutations in the male-sterile wheat plant;
b. growing plants from the mixture of wheat seeds, wherein Ms45 male-sterile wheat plants are female parent wheat plants, and wherein the wheat plants comprising the 4E chromosomal component are male parent plants;
C. allowing the male parent plants to fertilize the Ms45 male-sterile female parent wheat plants to produce wheat seeds comprising the homozygous mutations of the Ms45 male-fertility polynucleotide and wheat seeds comprising the homozygous mutations of the Ms45 male-fertility polynucleotide in the A, B, and D wheat genomes and the plant restoration donor chromosomal component.

31. The method of claim 30, comprising planting the mixture of wheat seeds in a row in the field.

32. The method of claim 30, comprising planting the mixture of wheat seeds in the same row in the field.

33. The method of claim 32, wherein at least about 20%, 25%, 30%, 35%, 40% or 45% of the wheat seeds produced are wheat seeds comprising the homozygous mutations of the Ms45 male-fertility polynucleotide and the 4E chromosomal component.

34. The method of claim 32, wherein at least about 55%, 60%, 65%, 70%, 75%, or 80% of the wheat seeds produced are wheat seeds comprising the homozygous mutations of the Ms45 male-fertility polynucleotide and not the 4E chromosomal component.

35. The method of claim 32, wherein the number of wheat seeds comprising the homozygous mutations of the Ms45 male-fertility polynucleotide produced is increased as compared the number of wheat seeds comprising the 4E chromosomal component and the homozygous mutations of the Ms45 male-fertility polynucleotide produced in a field where the wheat seeds that will develop into the female and male parent plants are planted in separate rows.

36. The method of claim 30, wherein the plant polynucleotide that confers the plant seed phenotype is located on the same chromosomal arm of the 4E chromosomal component as the Ms45 male-fertility restoration locus and not separated by a centromere.

37. (canceled)

38. The method of claim 30, wherein the 4E chromosomal component is from a non-wheat species.

39. (canceled)

40. The method of claim 30, wherein the male parent plant is a Blue Sando, Blue Baart, Blue Onas, Blue 1, PBB, or Blue Norco wheat line comprising the plant restoration donor chromosomal component from a non-wheat species.

41. (canceled)

42. (canceled)

43. The method of claim 30, wherein the second portion of seeds are wheat seeds comprising a euploid number of chromosomes, wherein one of the wheat chromosomes has been substituted with a chromosome comprising the plant restoration donor chromosomal component comprising the 4E chromosomal component from Thinopyrum or Agropyron.

44. The method of claim 43, wherein the chromosome comprising the plant restoration donor chromosomal component is substituted for a 5AL wheat chromosome.

45. The method of claim 30, wherein the plant polynucleotide that confers the plant seed phenotype is a phenotype for color, physiology, or morphology of the seed.

46. The method of claim 45, wherein the color is blue aleurone, P gene, anthocyanin, or Kala 4.

47. The method of claim 30, the method further comprising sorting the wheat seed into separate populations of wheat seeds based on the expression of the plant seed phenotype of the plant restoration donor chromosomal component, wherein the absence of the expression of the plant seed phenotype indicates the seed will produce male-sterile female wheat plants and wherein the presence of the expression of the plant seed phenotype indicates the seed will produce male-fertile wheat plants.

48. The method of claim 30, the method further comprising discarding any wheat seed homozygous for the plant restoration donor chromosomal component as indicated by increased expression level or concentration of the plant seed phenotype as compared to the expression level or concentration of the plant seed phenotype in wheat seed heterozygous for the plant restoration donor chromosomal component.

49.-136. (canceled)

137. The wheat plant cell of claim 13, wherein the 4E chromosomal component is a monosomic substitution for a wheat chromosome.

138. The wheat plant cell of claim 13, wherein the 4E chromosomal component is a monosomic substitution for a 5AL wheat chromosome.

139. The wheat plant cell of claim 13, wherein the 4E chromosomal component is part of a homoeologous chromosome pair, wherein the pair comprises a first and second chromosome, the first chromosome is native to the wheat plant cell and the second chromosome comprises the 4E chromosomal component.

140. The wheat plant cell of claim 13, wherein the homozygous Ms45 mutations are created by gene editing or chemical mutagenesis.

141. Wheat pollen produced from the wheat plant of claim 21.

142. The method of claim 30, wherein the homozygous Ms45 mutations are created by gene editing or chemical mutagenesis.
